SEQUENCE LISTING (3 sequences)

SEQ ID NO: 1 - DNA, 5696 bp, Homo sapiens
aatcgcgcacaatgagacagcgctgagcgccggaagtggggccgaaggaaaacacggagagggagcccggccgggacaggaagaggctggggaccgcggcgaaggtggtgagtgctcttgggcgccttctcccaacgtccctgccagactcgcctccgggctgattctccagttggtttcctggactccagagtagctgtccggcctggccccggaggtgcaaagtaagaaaattgaagtcaaagaccatgggagatacagcaaaaccttatttcgtgaagcgcactaaagaccgggggactatggatgatgatgacttcagaaggggtcacccccaacaagattatttaataatagatgaccatgctaaaggccatggcagtaaaatggaaaagggccttcaaaaaaagaagataacaccagggaactatgggaatacccccagaaaaggaccatgtgctgtttccagcaatccatatgcatttaaaaacccaatctacagtcaacccgcttggatgaatgacagccacaaagatcagagtaagagatggctgtctgatgaacatactggtaattcagacaactggagagaattcaaacctggacctagaattcctgttataaaccgacaaagaaaagactcctttcaagaaaatgaagatggttataggtggcaagacacaagaggctgcagaactgtaagacgactgtttcataaagacctaacaagcctagaaaccacgtcagaaatggaagcaggaagtcctgaaaacaagaagcagaggtccagacctaggaagccacggaagactagaaatgaggaaaatgagcaggatggagacttggaaggccctgtgatcgatgagtctgtactttcaacgaaggagctgctaggcttacagcaggctgaggagagactgaagagagactgcattgacaggctaaaaaggcgaccacgaaactaccctacagcaaagtacacctgcagactctgtgatgttttaattgaatccattgcatttgcccataagcatatcaaggaaaagaggcacaagaaaaacattaaggagaagcaagaggaagagttgctcactacgttacccccaccaacaccctcccagataaatgcagttggcattgccattgacaaagtggtacaggaatttggcttacacaatgagaacttggaacagaggctggaaattaaacgtatcatggaaaatgtgttccaacacaagttaccagattgttccctaagattatatgggtcatcctgtagcagattgggtttcaaaaattcggatgtaaacattgacatccagtttccagccattatgtctcagccagatgtcctcttacttgttcaagaatgtttaaagaacagtgactcctttattgatgttgatgcagacttccatgctagggtgccagtggtggtgtgcagagaaaagcaaagtggtcttctgtgtaaagtgagcgcaggaaatgaaaatgcttgtctgacaacaaagcatttaactgcccttggaaaactagaaccaaagctggttcctttggtgattgcatttaggtactgggcaaagctttgcagtatagatcgccctgaagaaggaggtctgccaccttatgtgtttgccctgatggccattttctttcttcagcagaggaaagaaccccttttgcctgtatatctaggatcatggattgaaggattctcattaagcaaactagggaatttcaaccttcaagacattgaaaaagatgttgtgatctgggaacatactgacagtgctgcaggggacacaggcataacaaaagaagaggcaccaagagaaacgccgattaaaaggggacaggtgtcattaatattggatgtgaaacaccagccttcagtaccagttgggcagctctgggtggaattgctgcggttctatgctttagaatttaatttggctgatttagtgataagtattcgtgtcaaagaattggtatctcgggaattgaaggattggcccaaaaagcgcattgccattgaagatccctactctgttaaaagaaatgtggcaagaaccctaaatagtcaacctgtgtttgaatatatacttcattgtttaaggacaacatacaagtattttgctcttccacacaaaattacaaaatccagccttctaaagcctctgaatgcaattacatgtatttcagaacattctaaagaagtaataaatcatcatccagatgtacaaacaaaagatgataagctcaaaaactcagttttggcccaaggtcctggtgctaccagttcagctgcaaatacctgtaaggtacagccacttactcttaaagagactgctgaaagttttggaagcccaccaaaagaagaaatgggaaatgaacacatcagtgtccaccctgaaaactcagactgtatccaagcagatgttaactctgatgattacaagggtgataaagtataccatccagaaacaggaaggaaaaacgagaaagagaaagttggaaggaagggcaagcatctgttgactgttgatcagaaacgtggagagcatgttgtctgtggcagcacacgtaataatgagtcagagagcactttggatttagaaggcttccaaaatcccacagctaaagagtgtgagggacttgccactttagataacaaggctgatcttgatggagaaagtacagaaggtactgaggaactagaagactctctaaaccactttacccactcagtacagggccagacatcagaaatgattccctctgatgaagaggaggaggacgacgaagaagaggaggaggaagaagaacctaggctcaccattaaccaaagggaagatgaagatggcatggctaatgaagatgagttagacaacacctacactgggtcaggggatgaggacgccctatctgaagaggatgatgagttaggcgaagctgctaagtatgaagacgtgaaagaatgtggaaaacatgtagaaagagctctcctagtggaacttaataaaataagtctcaaggaagaaaatgtatgtgaagaaaaaaattcacctgtggatcagtctgattttttttatgaattcagtaaacttatcttcaccaaaggcaagtctcctacggtagtgtgcagcttatgcaaacgagagggtcatctaaagaaggactgtcctgaagacttcaaaagaatccagctagaacctctgccaccattaacacccaagtttttaaatatcttagatcaagtctgtatccagtgttataaggatttttctccaacaattatagaagatcaggctcgtgaacatattcggcaaaacctagaaagtttcataagacaggactttccaggaactaaattgagcctgtttggctcctccaaaaatggatttgggttcaaacagagtgaccttgacgtctgtatgacaattaatggacttgaaactgctgagggattggactgtgtcagaactattgaagaattagcaagagtcctcagaaaacattcaggtctgagaaacatcttacctattacaacagcaaaggtgccaattgtgaagttcttccatttgagaagtggtctggaagtagatatcagtttgtataacacattggcccttcataacacaaggcttttatctgcttattccgccattgatcccagagtgaagtatttgtgctataccatgaaagtatttacaaagatgtgtgatattggtgatgcatctagaggcagcttatcatcgtatgcatatactcttatggtgctatattttctccagcagaggaatccaccagtcattcctgtccttcaagagatatacaaaggtgaaaagaaacctgaaatatttgttgatggctggaatatttatttttttgatcaaatagatgaactgcctacctattggtcagaatgtggaaaaaatacagaatctgttgggcagttatggttgggccttcttcgtttctacacagaggaatttgattttaaagaacatgttattagcatcaggagaaaaagtctgcttacaacttttaagaaacagtggacctcaaaatacattgttattgaagatccctttgatttgaatcataatcttggagctggattatcaaggaaaatgacaaattttataatgaaggcttttatcaatggtagaagagtatttggtattcctgtcaagggatttccaaaggactacccctcaaaaatggaatacttttttgatccagatgtgttaactgaaggagagctggccccaaatgatagatgttgtcgaatttgtggaaaaatcggacacttcatgaaggactgtcctatgaggagaaaagtaagacggcggcgagatcaggaagatgccctgaaccaaagataccctgagaacaaggaaaaaagaagcaaagaggacaaagaaattcacaacaagtacacagaaagggaggtgtcaacaaaagaagataagcccatacagtgcacacctcagaaagccaagccaatgcgggcagctgctgacctggggagggagaagatcctcaggccaccagtagaaaaatggaagagacaggatgacaaagacttaagagaaaaacgttgttttatttgtggaagagaagggcacattaaaaaggaatgcccacagtttaaaggctcttcaggtagcctttccagtaaatatatgactcagggaaaagcctcagcgaagaggacccagcaggaatcatgagggaaggaaaatgcagcactctaaatggccactcaggcgttcctattcactcggaaaattaggttcatttcacaggacacagcagtgtagatcaggcttcaacttaacatttaagggaaatgtcagatttttttttaatttaatgaaattgttaatgaggaaaaatttttaatatagtcttatctaccacacatccccatagatttaaggattttaatagaaagacatgatgtatgtatttaagccacgttaaaagaaaaaatataactatggaccggtattcagtgaatacagtttcatggtttttaattctttcaaagcacattaaaaatggtgtgctgataaaccccaagtaaattaaccctttttccgtataaatccattttttgttttgaagaggggaaattatatttattgttgtttactgaatcctggtgtgaaagcatatcagatatgtatgaactgctactgctgtacttccgatttacggacatcattttattgctatttgtagacgtgataacatgaacatgagtacctatttatgtgggccttcagtggatgggcagtgccactcaggtctctggggtttccctctctaattttaagtaaattgacatataactactatgcttataaaaatgaagtaaggaaaacaagtagtcctgtttgccactaaaaacattttcaaaggaaaaataaaatgaaagtactttttactttttatgatactcagaaattaggatgaagaacttttaaaattgctgaagatcaaagaggttatctctgccagtcacaagtgtggctggtgtcattctgggtctgactggagccctcctggactgtttctttaatttcaaaagccctgcagacatagtacctggtcagaactatgcctcggtttatttatcattttgaaataaaatcagaatttcaacctgtaaaaaaaaaaaaaaaaaa

SEQ ID NO: 2 - DNA, 1019 bp, Artificial Sequence
  gene-trap insertion in the Zcchc6 gene
tagacgcgcgtgctgcgttttccataggatmmgccccctgygagcwtcacaaaaaatcgacgctcagtcagaggtgcgaaaccgacagackataaaagatacagcgtttcccctggaagmtcctcgtgcgctctctgtccgmgctgcgcttacyggawacctgtccgccttctccttcgkgaagcrgtggcgctktctcatagctcacrcgtagrtatctcagttcggtgtaggtcgttcgctcaagctgggctgtgtgcacgaacycccygttcagccckaccgctgygccttrtmyggtaactatmgtcttgagtccaacccggtaagacacgactgatcgccactggcagcagccactggtaacaggattagcrgagcgaggtatgtaggcggtgcyacagagttcttgaagtggtggcstaastayggctacactagaagamcagtmtktkgtatctgcgctmtgsygaasccagwkaccttcgsaaaaagagttggtagctyytgatccggcamacaaaccaccgctsgtagcggtggtttttttgtttgcaagcagcagattacgcgcagaaaaaaaggatcmgcctrgtmtatataatgaagttccaggtcagccagkgstasatagtgagatcctgtctttgaaacaacaaacagattmtgaggtwggattccwygmtgttctysttcatmscctctccttccttccygtgcttaattctgtagctccctggttaggtagcccacgramwwwactgtttyttaaagaatttaattttagagtgtgtgtatgtgtgtgtstgttcatgtggstatmwgcacatgggyrcmggygtgccytcwsaggccagagtctccccagaagskggtatgacaagtgattgttaaayrtcccawgysggtgctgggaactaagctcgtgtcctctgsgaaacagtatgtgktyttacayytccagctccaatcttaatmttaytttaagtttcaaatttgatgtgatcaccagcagasat

SEQ ID NO: 3 - DNA, 966 bp, Artificial Sequence
  5′ RACE sequence for exons 2-6, Zcchc6
  misc_feature (721)..(721): n is a, c, g, or t
  misc_feature (891)..(891): n is a, c, g, or t
  misc_feature (930)..(930): n is a, c, g, or t
  misc_feature (932)..(932): n is a, c, g, or t
  misc_feature (935)..(936): n is a, c, g, or t
  misc_feature (958)..(959): n is a, c, g, or t
  misc_feature (964)..(966): n is a, c, g, or t
tgtatgctatacgaagtatcgatgcgatctgcgttcttcttctttggttttcgggacctgggacgacagctggaaactgaacatcaatgttcacgtcagagtctctgaagcccagtcggctacaggaggatccataaagcctaagagaacagtctggtagcttgtgccggaacacactttccatgacacgcttaatttctagtctctgatccaagttttcactgtgtaaaccaaactcctgcaccactctgtcaatggcgctgccaactgcatgtatctgggagggcgctggcgggggcagtgtggtgagcaattcttcctcctgcttttccttaaggttcttcttgtgcctcttctccttgatgtgcttatgaccacccgcaatggagtcaatcaaagcatcgcacagtttgcaggtatactttgctgtcgggcagtttcgtggtcgccttttcagcctgtcgatgcagtctctcttgagccgttcctcagcctgctggaggcccaacagctccttggttgaaaggacagactcgtcgatgacaggaccgtccagatctccatcctgctcactgtcctccgttctagtcctccgtggcttacgaggtctggaccgctgtttcttgttttcaggacttcctgcttccatttctgacatggcttctaggctgctgaggtctttcccccccccccccctctcacagctctgaagatctctccnactcctgccaccctgtaggcatcgtcactttcctgaaaggatttccttcctttgagcggctttatgacaggaaatcctaggtccgggcttgaactctctcccagcgtgtcctgcattgaccagcgagattcatcagcagtagccattttttattttggatctttgtggtnatcagttcatcccaagcaaggctgactggtaattgggtngnaanngcataaggaattgcctggcgannccccnnn

(19) United States
(12) Patent Application Publication
(10) Pub. No.: US 2019/0101533 A1
(43) Pub. Date: Apr. 4, 2019

(54) ANIMAL MODEL FOR STUDYING ZCCHC6 IN BONE DISEASE AND DEVELOPMENT

(71) Applicant: Northeast Ohio Medical University, Rootstown, OH (US)

(72) Inventor: Tariq M. HAQQI, Rootstown, OH (US)

(21) Appl. No.: 16/082,946

(22) PCT Filed: May 8, 2017
(86) PCT No.: PCT/US2017/031535

§ 371 Date: Sep. 7, 2018

Related U.S. Application Data
(60) Provisional application No. 62/304,563, filed on Mar. 7, 2016.

(51) Int. Cl. G01N 33/567; G01N 33/564; G01N 33/573; G01N 33/50; A01K 67/027; A61K 38/45

(52) U.S. Cl. CPC G01N 33/567; G01N 33/564; G01N 33/573; G01N 33/5088; A01K 67/0276; A01K 2267/03; G01N 2333/9125; G01N 2800/105; A01K 2217/054; A01K 2227/105; A61K 38/45

(57) ABSTRACT

A transgenic knockout non-human animal is described whose genome includes a heterozygous disruption of the expression of at least one endogenous gene encoding zinc-finger, CCHC domain-containing protein 6 (ZCCHC6). The transgenic animal model can be used to study bone disease or development by determining differences in bone characteristics, protein expression, or cytokine expression in the animal model in comparison to a corresponding wild-type non-human animal.

### CONTINUING APPLICATION DATA

This application claims the benefit of U.S. Provisional Application Ser. No. 62/304,563, filed Mar. 7, 2016, the disclosure of which is incorporated by reference herein.

### SEQUENCE LISTING

The instant application contains a Sequence Listing which has been submitted in ASCII format via EFS-Web and is hereby incorporated by reference in its entirety. Said ASCII copy, created on May 2, 2017, is named ZCCHC6 model_ST25 and is 11,212 bytes in size.

### BACKGROUND

Skeletal development and homeostasis are tightly regulated and coupled processes. Bone is a dynamic tissue that undergoes constant remodeling. Osteoblasts (OBs) arise from a mesenchymal stem cell progenitor that first deposit a layer of organic bone matrix followed by a layer of hydroxyapatite minerals. Osteoclasts (OCs) are large multinucleated cells responsible for bone resorption and degradation. Dysfunction in bone remodeling can have severe effects in development and can lead to bone pathology.

Mechanisms associated with intrinsic RNA regulation are important players in development and disease processes. RNA tailing modifies small RNA molecules by the addition of untemplated nucleotides to the 3′ end of RNA. One of the most widely studied family of enzymes involved in RNA tailing are the canonical poly(A) polymerases (PAPs), which are responsible for the generation of poly(A) tails and promoting RNA stability. Several non-canonical PAPs have also been described with the nucleotidyl transferase activity. Rissland et al., Mol Cell Biol 27(10):3612-3624 (2007). The terminal uridylyl transferases (TUT/Zcchc) are a family of non-canonical PAPs that catalyze the uridylation and in some instances, the adenylation of RNA substrates; however their role in bone development and disease is poorly understood. Norbury C J, Nature reviews. Molecular cell biology 14(10):643-653 (2013).

Osteoarthritis (OA) is a global disease which affects the whole joint, affects the quality of life, and has a complex etiology with many factors—some modifiable—that contribute to an increased risk of OA and include obesity, genetics, aging and trauma to the joint. When clinically evident, OA is characterized by joint pain, tenderness, limitation of movement, crepitus, occasional effusion, and variable degrees of inflammation without systemic effects. OA pathology is now being recognized as driven by a pro-inflammatory component as high levels of inflammatory cytokines are present in the synovial fluid of patients with OA, and also seen in animal models of OA. Haseeb A and Haqqi T M., Clin Immunol., 146(3):185-96 (2013).

Over 27 million Americans suffer from OA, which causes joint dysfunction leading to a compromised quality of life. Clinical options in OA are currently limited to the use of non-steroidal anti-inflammatory drugs (NSAIDS) but ultimately the affected joint is replaced by the total joint arthroplasty (TJA) procedure. However, TJA is expensive and only carried out as a last resort. From the onset of disease, OA patients suffer acute pain and become disabled due to disease progression resulting in the loss of joint function. Furthermore, the disease is a multifactorial process that is impacted by aging, genetic predisposition, abnormal biomechanics, obesity, and trauma. Accordingly, there remains a significant need to develop an understanding OA so that additional approaches for treating OA and related diseases can be developed.

### SUMMARY OF THE INVENTION

TUT7 (also known as ZCCHC6), a non-canonical poly(A) polymerase, has been shown to be important for the regulation of multiple functions in immune cells but its expression and function in bone is not known. The inventors investigated the expression and role of TUT7 in bone homeostasis in mice.

TUT7 knockout mice were generated by GeneTrapping. Gene expression was determined by qPCR. Bone mass and matrix mineralization were determined by μCT, DEXA and von Kossa staining. Osteocalcin, CTX-I, RANKL and OPG levels were determined by ELISA. Osteoblast and osteoclast differentiation were assessed by ALP and TRAP staining. Analysis of Osteoblast-mediated osteoclastogenesis was performed using co-culture assay. Protein expression was determined by Western immunoblotting.

TUT7 was expressed in osteoblasts, osteoclasts but the expression decreased with age in bone. Mice with global TUT7 deficiency exhibit higher bone mineral density, cancellous bone volume and enhanced osteoblast differentiation and function ex vivo compared to wild type littermates. Mineral apposition and bone formation rate were significantly high in TUT7 deficient mice. Osteoclast number and bone resorption were significantly reduced in TUT7KO mice. TUT7KO mice had low serum RANKL but increased expression of Osterix in osteoblasts. Importantly, reintroduction of TUT7 inhibited the Osterix expression and activity in TUT7KO osteoblasts.

The inventors conclude that TUT7 is a novel, negative regulator of bone mass and acts through the inhibition of Osterix expression and activity in osteoblasts. The findings demonstrate a previously unrecognized role of TUT7 in bone homeostasis that is not compensated for by other members of the family.

### BRIEF DESCRIPTION OF THE FIGURES

The present invention may be more readily understood by reference to the following figures, wherein:

FIGS. 1A-1H provide graphs and images showing that the absence of TUT7 expression has no severe effect on early postnatal development and skeletal morphology. (A) Differential mRNA expression of TUT7 in different tissues in WT mice as determined by SYBR-Green based qPCR. TUT7 was widely expressed in different tissues including bone. (B) Expression of TUT7 mRNA in aging long bone tissue in WT mice. TUT7 mRNA expression was highest during early postnatal skeletal development. (C) Generation of TUT7 KO mice was used by gene trapping. PCR of genomic DNA used to confirm wildtype (+/+; WT), heterozygous (+/−), and homozygous (−/−; TUT7KO). (D) Confirmation of the absence of TUT7 by qPCR in different tissues. (E-F) Absence of TUT7 had no effect on body weight or body length. Measurements of body weights (E) and lengths (F) of male and female WT and TUT7KO mice during different stages of development. (G-H) Genomic deletion of TUT7 had no apparent effect on skeletal morphology. Skeletal prep (G) of 5-day old WT and TUT7KO mice and X-ray analysis (H) of 8-week old WT and TUT7KO female mice. Data are expressed as means+/−SEM. ***P<0.001.

FIGS. 2A-2H provide graphs and images showing that TUT7 deficiency enhances bone formation in vivo. (A) Absence of TUT7 had no effect on the lengths of WT and TUT7KO femurs at 4-, 8-, and 16-weeks of age. (B-C) Ablation of TUT7 results in enhanced bone mineral density (BMD; g/cm²) as shown in WT and TUT7KO male and female full body (B) and femur (C) by DEXA analysis. (D-H) Absence of TUT7 enhances trabecular bone mass compared to WT. MicroCT 3D-images (D) and analysis (E-H) of WT and TUT7KO male and female 8-week femurs. (E-H). Notice the increase in bone volume/tissue volume (BV/TV) (E), trabecular number (Tb.N) (F) and trabecular thickness (Tb.Th) (H) and decrease in trabecular separation (Tb.Sp.) (G) seen in the TUT7KO mice and compared to WT mice. Data are expressed as means+/−SEM. *P<0.05, **P<0.01.

FIGS. 3A-3H provide graphs and images showing the absence of TUT7 expression increases bone matrix mineralization in vivo. (A-E) Histological images (A) and analyses (B) of femurs from 8-weeks old WT and TUT7KO mice. Deficiency of TUT7 expression results in enhanced trabecular number (Tb.N) and decreased trabecular separation (Tb.Sp.), but no difference in the number of osteoblasts (N.Ob/B.Pm) or osteoblast surface (Ob.S/BS) (B). (C-D) Dynamic histomorphometric calcein images (C) and analyses (D) of femurs from 8-weeks old WT and TUT7KO mice. Notice the significant increase in the mineral apposition rate (MAR) in TUT7KO mice and bone formation rate (BFR) compared to WT mice (D). (E) Osteocalcin levels in the serum of WT and TUT7KO mice determined by ELISA. (F) Expression of TUT7 in WT bone-marrow progenitor cells during osteoblast differentiation. TUT7 is most highly expressed during early osteoblastogenesis. (G-H) Absence of TUT7 enhances osteoblast differentiation. Bone-marrow progenitor cells from WT and TUT7KO mice were differentiated with osteogenic media or left undifferentiated, and terminated for ALP staining, activity, and qPCR (G) analyses. Furthermore, bone-marrow progenitor cells differentiated with osteogenic media were terminated for von Kossa staining and analyses (H). Scale bar; 200 μm. Data are expressed as means+/−SEM. *P<0.05, ***P<0.01.

FIGS. 4A-4G provide graphs and images showing that TUT7 deficiency inhibits osteoclast differentiation and resorption in vivo, but has no effect on osteoclastogenesis in vitro. (A-B) TRAP stained images (A) and histomorphometric analyses (B) of femurs from 16-weeks old WT and TUT7KO mice. Absence of TUT7 expression significantly decreased the number of osteoclasts per bone perimeter (N.Oc/B.Pm), but has no effect on the percentage of osteoclast surface per bone surface (Oc.S/BS) (B). (C-D) Biochemical analyses of osteoclast markers in serum of 16-weeks old WT and TUT7KO mice. Notice the significant reduction in CTX-1 (C), RANKL, and RANKL/OPG ratio, but no difference in the levels of OPG (D). TUT7 expression during osteoclastogenesis (E). TUT7 was not required for osteoclast differentiation in vitro. Osteoclast bone marrow progenitor cells from WT and TUT7KO mice were plated, differentiated, and terminated for TRAP staining and analysis (F). Ablation of TUT7 has no effect on osteoclast resorption activity in vitro. Osteoclast bone marrow progenitor cells (OCPs) from WT and TUT7KO mice were plated on Corning calcium phosphate discs, differentiated, and terminated to analyze resorption activity and to quantify the resorption area (G). Scale bar; 200 μm. Data are expressed as means+/−SEM. *P<0.05.

FIGS. 5A-5F provide graphs and images showing that osteoblasts from TUT7KO mice inhibit osteoclastogenesis by inhibiting RANKL expression. (A-C) Co-culture assay and analyses of WT or TUT7KO mice-derived primary calvaria osteoblasts cultured with bone marrow-derived osteoclast progenitor cells from WT or TUT7KO mice. Note that in cultures containing TUT7KO-derived osteoblasts the number of WT mouse-derived osteoclasts was significantly reduced compared to co-cultures with WT osteoblasts as shown by TRAP staining (A), activity (B), and count (C). (D-E) TUT7 deficiency inhibits RANKL expression in osteoblasts, but not of OPG mRNA expression. Total RNA isolated from undifferentiated and differentiated WT and TUT7KO osteoblasts was used to examine the expression of RANKL (D) and OPG (E) mRNAs by qPCR. (F) Absence of TUT7 inhibits RANKL expression in long bone and calvaria of TUT7KO mice as determined by qPCR. Scale bar; 200 μm. Data are expressed as means+/−SEM. *P<0.05, **P<0.01, ***P<0.01.

FIGS. 6A-6H provide graphs and images showing that TUT7 negatively regulates Osterix expression in osteoblasts. (A-B) Osteogenesis gene expression analyses in osteoblasts from WT and TUT7KO mice revealed a 7-fold increase in Osterix mRNA expression in the osteoblasts from TUT7KO mice and was verified by qPCR (C). Removal of TUT7 significantly enhances Osterix protein expression in osteoblasts (D). (E-F) Overexpression of TUT7 negatively regulates the expression of osteoblast markers in vitro. MC3T3-E1 osteoblast-like cells overexpressing TUT7 showed a significant reduction in Osterix mRNA expression and ALP and a significant increase in RANKL expression as determined by qPCR (E). Protein isolated from MC3T3-E1 osteoblast-like cells overexpressing TUT7 showed a significant reduction in the levels of Osterix protein by Western immunoblotting (F). (G-H) Overexpression of TUT7 negatively regulates Osterix activity in vitro. MC3T3-E1 osteoblast-like cells overexpressing TUT7 showed a significant reduction in Osterix activity determined using a Luciferase-based reporter vector (G). Primary osteoblasts from WT mice overexpressing TUT7 also resulted in a significant reduction in Osterix activity; however, when TUT7 expression was restored in TUT7KO osteoblasts, Osterix activity was downregulated and was similar to the levels in WT osteoblasts as determined by luciferase activity assay (H). Data are expressed as means+/−SEM. *P<0.05, **P<0.01.

FIG. 7 provides images showing chondrocytes present in the damaged human cartilage express IL-6 (arrows). IL-6 expressing chondrocytes were immunohistochemically localized using a mouse monoclonal antibody (sc-130326) and the VectaStain kit. IL-6 expressing chondrocytes were abundant in the damaged area (no Safranin-O staining). Control sections were incubated with mouse isotype control IgG only. SC-smooth cartilage, DC-damaged cartilage.

FIGS. 8A-8C provide graphs and images showing IL-1β is a potent inducer of IL-6 and ZCCHC-6 expression in OA chondrocytes. Chondrocytes were stimulated with IL-1β (5 ng/ml) for 24 h (A) IL-6 gene expression was determined using Taqman assay and is shown relative to the expression levels of housekeeping gene β-actin. (B) Secreted IL-6 was quantified by sandwich ELISA. (C) ZCCHC6 protein expression was determined using Western immunoblotting. Representative results are shown. Data are represented as Mean±SD (n=3). Each assay was run in duplicate. *p<0.005

FIGS. 9A and 9B provide images and a graph showing ZCCHC6 knockdown inhibited IL-6 expression in IL-1β-stimulated human chondrocytes. (A) SMARTpool siRNA-induced knockdown of ZCCHC6 was confirmed by immunoblotting. (B) Human chondrocytes transfected with non-targeting and ZCCHC6-targeting SMARTpool siRNAs were stimulated 48 h later with IL-1β (1 ng/ml) for 3 h, 6 h, and 24 h and secreted IL-6 protein concentration in culture supernatants was determined by sandwich ELISA, n=3, *p<0.005, **p<0.001, ZCCHC6 siRNA transfected chondrocytes vs. chondrocytes transfected with native control siRNAs. NC=Negative Control.

### DETAILED DESCRIPTION OF THE INVENTION

The present invention relates to a transgenic knockout non-human animal whose genome includes a heterozygous disruption of the expression of at least one endogenous gene encoding zinc-finger, CCHC domain-containing protein 6 (ZCCHC6). The transgenic animal model can be used to study bone disease or development by determining differences in bone characteristics, protein expression, or cytokine expression in the animal model in comparison to a corresponding wild-type non-human animal.

### Definitions

One embodiment of the present invention is directed to an animal, preferably, a rodent, more preferably, a mouse, comprising in its germline cells (embryonic stem cells or germ cells) an artificially induced heterozygous ZCCHC6 gene deficiency mutation.

The phrase “non-human animal,” includes non-human vertebrate animal, and more preferably a mammal, such as a domesticated farm animal (e.g., cow, horse, pig), pet (e.g., dog, cat), or rodent. The terms “rodent” or “rodents” refers to any and all members of the phylogenetic order Rodentia (e.g., mice, rats, squirrels, beavers, woodchucks, gophers, voles, marmots, hamsters, guinea pigs, and agoutas) including any and all progeny of all future generations derived therefrom. The term “murine” refers to any and all members of the family Muridae, including, but not limited to, rats and mice.

By “heterozygous disruption” is meant a mutation of an embryonic stem cell/germ cell or animal, wherein one allele of the endogenous gene (such as ZCCHC6) has been disrupted, such that the translation product(s), which is/are typically expressed in cells bearing the wild-type genotype, is/are not expressed or is/are not functional in at least one aspect in cells of the targeted organism. By “knockout” or “KO” is meant having all or part of a gene eliminated or inactivated/deactivated by genetic engineering.

By “functional,” when used herein as a modifier of ZCCHC6 protein(s), peptide(s), or fragments thereof, refers to a protein/polypeptide that exhibits at least one of the functional characteristics or biological activities attributed to ZCCHC6.

By “transgenic” or “recombinant” animal is meant an animal that has had foreign or exogenous DNA introduced into its germ line cells, e.g., embryonic stem (ES) cells or germ cells. The exogenous genes which have been introduced into the animal's cells are called “transgenes” or “recombinants” The introduction or insertion of foreign DNA is also termed transfection. Preferably, the transfected germ line cells of the transgenic animal have the non-endogenous (exogenous) genetic material (such as a targeting vector) integrated into their chromosomes. ES cell line used according to the present invention is selected for its ability to integrate into and become part of the germ line of a developing embryo so as to create germ line transmission of the transgene or targeting vector. Those skilled in the art will readily appreciate that any desired traits generated as a result of changes to the genetic material of any transgenic animal produced by the present invention are heritable. Although the genetic material may be originally inserted solely into the germ cells of a parent animal, it will ultimately be present in the germ cells of direct progeny and subsequent generations of offspring. The genetic material is also present in the differentiated cells, i.e. somatic cells, of the progeny.

The term “progeny” or “offspring” refers to animals of any and all future generations derived or descending from a particular animal, e.g., a mouse ancestor or chimeric mouse containing one or more targeting vectors inserted or integrated into its genomic DNA, whether the animal is heterozygous or homozygous for the targeting vector. However, according to the present invention, homozygous ZCCHC6 is lethal. Progeny of any successive generation are included herein such that the progeny generations, i.e., the F1, F2, F3 and so on, containing the targeting vector are encompassed by this definition.

By “targeting vector” is meant a polynucleotide sequence that is designed to suppress or, preferably, eliminate expression or function of a polypeptide encoded by an endogenous gene in one or more cells of an animal. The polynucleotide sequence used as the targeting vector is typically comprised of (1) DNA from a portion or certain portions of the endogenous gene (e.g., one or more exon sequences, intron sequences, and/or promoter sequences) to be suppressed and (2) a selectable marker sequence used to detect the presence of the targeting vector in a cell. In some embodiments, the targeting vector is a gene trapping vector. The targeting vector is artificially introduced into a cell containing the endogenous gene to be mutated or disrupted (e.g., the ZCCHC6 gene). The targeting vector can then integrate within one or both alleles of the endogenous ZCCHC6 gene, and such integration of the ZCCHC6 targeting vector can prevent or interrupt transcription of the full-length endogenous ZCCHC6 gene or its subunit(s). Integration of the ZCCHC6 targeting vector into the cellular chromosomal DNA is typically accomplished via homologous recombination (i.e., regions of the ZCCHC6 targeting vector that are homologous or complimentary to endogenous ZCCHC6 DNA sequences can hybridize to each other when the targeting vector is inserted into the cell; these regions can then recombine so that the targeting vector is incorporated into the corresponding position of the endogenous DNA).

A gene trapping vector, as used herein, is a targeting vector including a downstream transcriptional termination sequence (e.g., a polyadenylation sequence; polyA). When inserted into an intron of an expressed gene, the gene trap cassette is transcribed from the endogenous promoter of that gene in the form of a fusion transcript in which the exon(s) upstream of the insertion site is spliced in frame to the reporter/selectable marker gene. Since transcription is terminated prematurely at the inserted polyadenylation site, the processed fusion transcript encodes a truncated and nonfunctional version of the cellular protein and the reporter/selectable marker is expressed.

The terms “protein”, “peptide”, and “polypeptide” are used interchangeably herein. As used herein, a “promoter” or “promoter region” refers to a segment of DNA that controls transcription of a DNA polynucleotide to which it is operatively linked. The promoter region includes specific sequences that are sufficient for RNA polymerase recognition, binding and transcription initiation. In addition, the promoter region includes sequences that modulate this recognition, binding and transcription initiation activity of RNA polymerase. These sequences can be cis acting or can be responsive to trans acting factors. This term also includes polypeptides that have been modified or derivatized, such as by glycosylation, acetylation, phosphorylation, and the like.

By “expression” is meant a process by which polynucleic acids are transcribed into mRNA and translated into peptides, polypeptides, or proteins. If the polynucleic acid is derived from genomic DNA and an appropriate eukaryotic host cell or organism is selected, expression can include splicing of the mRNA. The “nucleic acid” encompasses ribonucleic acid (RNA) or deoxyribonucleic acid (DNA), which DNA can be complementary DNA (cDNA) or genomic DNA, e.g. a gene encoding a ZCCHC6 protein. “Polynucleotides” encompass nucleic acids containing a “backbone” formed by phosphodiester linkages between ribosyl or deoxyribosyl moieties.

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native inter-nucleoside bonds, or both. The nucleic acid can be in any topological conformation. For instance, the nucleic acid can be single-stranded, double-stranded, triple-stranded, quadruplexed, partially double-stranded, branched, hair-pinned, circular, or in a padlocked conformation.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a peptide” also includes a plurality of such peptides.

### ZCCHC6 Knockout Animal Model

In one aspect, the present invention provides a transgenic knockout non-human animal whose genome comprises a heterozygous disruption of the expression of at least one endogenous gene encoding zinc-finger, CCHC domain-containing protein 6 (ZCCHC6).

ZCCHC6 (zinc-finger, CCHC domain-containing protein 6; a.k.a. TUT7) is a TUTase and member of the DNA polymerase β-like (pol-β) nucleotidyltransferase superfamily and has recently been shown to be involved in the biogenesis of let-7 miRNAs. Thornton et al., RNA 18:1875-1885 (2012). ZCCHC6, also known as PAPD6 (PAP associated domain containing 6) or HS2, is a 1,495 amino acid uridyltransferse that mediates RNA uridylation. Schmidt M-J and Norbury C J., WIREs RNA 1:142-151 (2010). ZCCHC6 protein contains three CCHC-type zinc fingers and two PAP-associated domains but their physiological significance has not yet been established. The gene encoding ZCCHC6 maps to human chromosome 9q21.33 and mouse chromosome 13 B2. Zinc-finger proteins contain DNA-binding domains and have a wide variety of functions, most of which encompass some form of transcriptional activation or repression. Several recent studies have implicated TUTases in the regulation of pre-miRNAs and mature miRNAs through uridylation or adenylation of their 3′ends. Ameres S L and Zamore P D, Nat Rev Mol Cell Biol. 14:475-488 (2013). For example, ZCCHC11 oligouridylates pre-let-7 miRNA in embryonic stem cells and masks the two nucleotides 3′ overhang of the pre-miRNA and prevents substrate recognition by Dicer, thus blocking the miRNA maturation. Hagan et al., Nature Struct Mol Biol. 16:1021-1025 (2009). ZCCHC11 which is longer than ZCCHC6 by 149 amino acids and efficiently incorporates both cytosines and uridines into RNAs has also been implicated in targeting human histone mRNAs for degradation. Jones et al., Nat Cell Biol., 11:1157-1163 (2009). ZCCHC11 also adds uridine to mature miR-26a and miR-26b which abrogates IL-6 repression and facilitates the high level expression of IL-6 in A549 and MLL cells. In preliminary studies, the inventors found that ZCCHC6, a member of the same family of enzymes as ZCCHC11, but is shorter and almost exclusively incorporates only uridines in RNA, is expressed at high levels in chondrocytes present in the damaged human OA cartilage and was mostly localized in the cytoplasm. Expression of ZCCHC11 was marginally upregulated by treatment of human chondrocytes with IL-1β and its knockdown did not alter IL-6 mRNA expression, suggesting that ZCCHC11 may not be involved in regulating IL-6 expression in IL-1β-stimulated primary human chondrocytes. SEQ ID NO: 1 provides the nucleotide sequence for Homo sapiens ZCCHC6 transcript variant 1 (mRNA).

In accordance with the present invention, a gene deficiency or heterozygous disruption of a gene is artificially induced. Artificial induction of such mutation can be accomplished by any means now known in the art or later developed. This includes well-known techniques such as homologous recombination, transpositional recombination, site-directed mutation, and artificial induction of frame shift mutations. A preferred method is homologous recombination.

In accordance of the present invention, an animal, preferably, a rodent, more preferably, a mouse, can be artificially mutated in at least one of the endogenous ZCCHC6 alleles, whereby the germ line cells of said animal lack the ability to express functional ZCCHC6 protein. Such mutation can be accomplished by various means known in the art, including, but not limited to, homologous recombination, transpositional recombination, site directed mutation, and a frame shift mutation within a region or regions of the ZCCHC6 gene crucial to expression of a functional ZCCHC6 polypeptide. Typically, such mutation is introduced into an embryonic stem cell (ES) (see Examples below) or a germ cell, such as an oocyte or male germ cell, which is then used to produce a transgenic zygote by mating with a germ cell of the opposite sex.

Where the ZCCHC6 targeting vector is transfected into the genome of a germ cell, the targeted germ cell then can be combined with a germ cell of the opposite sex-which also can be transfected with a targeting vector-in order to obtain a zygote. The uptake of an exogenously supplied nucleic acid segment, such as a targeting vector, will reach male germ cells that are at one or more developmental stages, and will be taken up by those that are at a more receptive stage. The primitive spermatogonial stem cells, known as AO/As, differentiate into type B spermatogonia. The latter further differentiate to form primary spermatocytes, and enter a prolonged meiotic prophase during which homologous chromosomes pair and recombine. Several morphological stages of meiosis are distinguishable: preleptotene, leptotene, zygotene, pachytene, secondary spermatocytes, and the haploid spermatids. The latter undergo further morphological changes during spermatogenesis, including reshaping of their nuclei, the formation of acrosome, and assembly of the tail. The final changes in the spermatozoon take place in the genital tract of the female, prior to fertilization. The male germ cells can be modified in vivo using gene therapy techniques, or in vitro using a number of different transfection strategies. See WO 00/69257.

In a preferred embodiment, the mutation is introduced by homologous recombination between at least one of the cell's endogenous copies of the ZCCHC6 gene and a targeting vector, where the targeting vector is transfected into the ES cell's genome. The ES cell then can be injected into a blastocyst, microinjected into a C57BL/6J blastocyst. The resulting recombinant blastocyst or zygote, as the case may be, can be implanted into a pseudopregnant host, representing the F0 generation. The F1 progeny then can be screened for the presence of one or more mutant ZCCHC6 allele. For example, according to the present invention, F1 animals can be produced by mating chimeric males (having the transgene) with C57BL/6 females. ZCCHC6 chimeras can be confirmed by genomic analysis techniques known in the art, such as, e.g., Southern blotting. The confirmed heterozygous animals, e.g., mice, are then intercrossed or mated to generate F2 animals. In accordance with the present invention, the F2 animals can be backcrossed to wild animals of the same species for sufficient generations, preferably, for two or more generations, more preferably, for five or more generations, and fed with appropriate diet. For example, the F2 mice of the present invention are backcrossed with C57BL/6 mice for five generations. All phenotypic characterizations are performed with wild-type (+/+) and heterozygous (+/−) within the same generation, all animals 10 to 12 weeks old.

In a preferred embodiment, the ZCCHC6 heterozygous disruption mutant animal can be generated by homologous recombination with a targeting vector as follows:

A ZCCHC6 targeting vector typically is prepared by isolating a genomic ZCCHC6 or cDNA ZCCHC6 polynucleotide sequence fragment and inserting a selectable genetic marker, typically comprised of an exogenous polynucleotide sequence, into said genomic or cDNA ZCCHC6 fragment. The ZCCHC6 gene or gene fragment to be used in preparing the targeting vector can be obtained in a variety of ways.

A naturally occurring genomic ZCCHC6 polynucleotide sequence fragment or cDNA molecule to be used in preparing the targeting vector can be obtained using methods well known in the art such as described by Sambrook et al. (Molecular Cloning: A Laboratory Manual, Cold Spring Harbor Laboratory Press, Cold Spring Harbor, N.Y., 1989). Such methods include, for example, PCR amplification of a particular DNA polynucleotide sequence using oligonucleotide primers, or screening a genomic library prepared from cells or tissues that contain the ZCCHC6 gene with a cDNA probe encoding at least a portion of the same or a highly homologous ZCCHC6 gene in order to obtain at least a portion of the ZCCHC6 genomic polynucleotide sequence. Alternatively, if a cDNA sequence is to be used in a targeting vector, the cDNA can be obtained by screening a cDNA library (preferably one prepared from tissues or cells that express the ZCCHC6 genomic sequence, where the tissues or cells are derived from the same or similar species of mammal as the targeted species) with oligonucleotide probes, homologous cDNA probes, or antibodies (where the library is cloned into an expression vector).

The ZCCHC6 genomic DNA fragment or ZCCHC6 cDNA molecule prepared for use in the targeting vector should be generated in sufficient quantity for genetic manipulation. Amplification can be conducted by 1) placing the fragment into a suitable vector and transforming bacterial or other cells that can rapidly amplify the vector, 2) by PCR amplification, 3) by synthesis with a DNA synthesizer, or 4) by other suitable methods.

The genomic ZCCHC6 polynucleotide sequence fragment, cDNA molecule, or PCR-generated fragment for incorporation into the ZCCHC6 targeting vector (referred to herein as “the ZCCHC6 polynucleotide sequence portion of the targeting vector”) can be digested with one or more restriction endonucleases selected to cut at a restriction site(s) also present in the selectable marker sequence, such that the selectable marker sequence can be inserted into a desired position within the ZCCHC6 polynucleotide sequence portion of the targeting vector. That is, the selectable marker sequence is inserted into a position along the ZCCHC6 polynucleotide sequence portion of the targeting vector, such that, were the selectable marker sequence inserted into the chromosomal copy of the ZCCHC6 gene of a particular cell that typically expresses ZCCHC6 protein, functional ZCCHC6 protein would not be expressed in said cell. The particular position will vary depending on a number of factors, including the available restriction sites in the ZCCHC6 polynucleotide DNA sequence fragment into which the selectable marker sequence is to be inserted, whether an exon sequence or a promoter sequence, or both is (are) to be interrupted, and whether several isoforms exist in the mammal (due to alternative splicing) and only one such isoform is to be disrupted. After the ZCCHC6 polynucleotide sequence portion of the targeting vector has been digested and the selectable marker sequence inserted therein, the selectable marker sequence should be flanked by at least about 600, preferably, about 1,000, polynucleotide base pairs remaining from the digested ZCCHC6 polynucleotide sequence portion of the targeting vector. This way, the flanking portions can hybridize with a targeted chromosomal ZCCHC6 gene on either side of the desired site of insertion of the selectable marker sequence into the chromosomal ZCCHC6 gene. In any event, the exogenous selectable marker sequence should be flanked by polynucleotide sequences, complimentary to the sense strand of the chromosomal ZCCHC6 gene, that are of sufficient length to facilitate hybridization with the targeted chromosomal ZCCHC6 gene, in order to achieve the desired homologous recombination between nucleotides in the targeting vector and at least one copy of the chromosomal copy of the ZCCHC6 gene.

For example, in some embodiments, expression of the ZCCHC6 is disrupted by recombination between the endogenous ZCCHC6 gene and a ZCCHC6 gene targeting vector comprising SEQ ID NO: 2.

The selectable marker sequence used in the targeting vector can be any nucleic acid molecule that is detectable and/or assayable after it has been incorporated into the genomic DNA of an ES or germ cell, and ultimately the heterozygous disruption animals. Expression or presence in the genome or lack thereof can easily be detected by conventional means, as further described herein. Preferably, the selectable marker sequence encodes a polypeptide that does not naturally occur in the animal. The selectable marker sequence is usually operably linked to its own promoter or to another strong promoter, such as the thymidine kinase (TK) promoter or the phosphoglycerol kinase (PGK) promoter, from any source that will be active or can easily be activated in the cell into which it is inserted; however, the selectable marker sequence need not have its own promoter attached, as it can be transcribed using the promoter of the gene to be mutated. In addition, the selectable marker sequence will normally have a polyA sequence attached to its 3′ end; this sequence serves to terminate transcription of the selectable marker sequence. Preferred selectable marker sequences are any antibiotic resistance gene, such as neo (the neomycin resistance gene), or a bacterial gene, such as beta-gal (beta-galactosidase).

After the ZCCHC6 polynucleotide sequence portion of the targeting vector has been digested with the appropriate restriction enzyme(s), the selectable marker sequence molecule can be ligated with the ZCCHC6 polynucleotidal sequence portion of the targeting vector using methods well known to the skilled artisan. In some cases, it is preferable to insert the selectable marker sequence in the reverse or antisense orientation with respect to the ZCCHC6 nucleic acid sequence; this reverse insertion is preferred where the selectable marker sequence is operably linked to a particularly strong promoter.

The ends of the DNA molecules to be ligated must be compatible; this can be achieved by either cutting all fragments with those endonucleases that generate compatible ends, or by blunting the ends prior to ligation. Blunting can be done using methods well known in the art, such as for example by the use of Klenow fragments (DNA polymerase I) to fill in sticky ends. After ligation, the ligated constructs can be screened by selective restriction endonuclease digestion to determine which constructs contain the marker sequence in the desired orientation.

The ligated DNA targeting vector then can be transfected directly into embryonic stem cells (see Example) or germ cells, or it can first be placed into a suitable vector for amplification prior to insertion.

The ZCCHC6 targeting vector is typically transfected into stem cells derived from an embryo (embryonic stem cells, or “ES cells”). ES cells are undifferentiated cells that are capable of differentiating into and developing into all cell types necessary for organism formation and survival. Generally, the ES cells used to produce the heterozygous disruption animal will be of the same species of animal as the heterozygous disruption animal to be generated. Thus for example, mouse embryonic stem cells will usually be used for generation of ZCCHC6 heterozygous disruption mice.

The embryonic stem cell line used is typically selected for its ability to integrate into and become part of the germ line of a developing embryo so as to create germ line transmission of the targeting vector. Thus, any ES cell line that is believed to have this capability is suitable for use herein. Preferred ES cell lines for generating heterozygous disruption mice are murine ES cell line AEO325. The cells are cultured and prepared for DNA insertion using methods well known to the skilled artisan, such as those set forth by Robertson (Teratocarcinomas and Embryonic Stem Cells: A Practical Approach, E. J. Robertson, ed. IRL Press, Washington, D.C., 1987), Bradley et al. (Current Topics in Devel. Biol., 20:357-371 (1986)) and Hogan et al. (Manipulating the Mouse Embryo: A Laboratory Manual, Cold Spring Harbor Laboratory Press, Cold Spring Harbor, N.Y., 1986).

Insertion (also termed “transfection”) of the targeting vector into the embryonic stem (ES) cells or germ cells can be accomplished using a variety of methods well known in the art including for example, electroporation, microparticle bombardment, microinjection, viral transduction, and calcium phosphate treatment (see Robertson, ed., supra). A preferred method of insertion is electroporation.

The ZCCHC6 targeting vector to be transfected into the cells can first be linearized if the targeting vector has previously been inserted into a circular vector. Linearization can be accomplished by digesting the DNA with a suitable restriction endonuclease selected to cut only within the vector sequence and not within the targeting vector sequence.

The isolated ZCCHC6 targeting vector can be added to the ES cells or germ cells under appropriate conditions for the insertion method chosen. Where more than one targeting vector is to be introduced into the cells, the DNA molecules encoding each such vector can be introduced simultaneously or sequentially. Optionally, heterozygous ZCCHC6 disruption ES cells can be generated by adding excessive ZCCHC6 targeting vector DNA to the cells, or by conducting successive rounds of transfection in an attempt to achieve homologous recombination of the targeting vector on both endogenous ZCCHC6 alleles.

Preferably, the ES cells or germ cells are electroporated for introduction of the transgene or ZCCHC6 targeting vector. The cells and targeting vector DNA are exposed to an electric pulse using an electroporation machine and following the manufacturer's guidelines for use. After electroporation, the cells are typically allowed to recover under suitable incubation conditions. The cells are then screened for the presence of the targeting vector.

Screening the transfected cells can be accomplished using a variety of methods, preferably, by screening the presence of the selectable marker sequence portion of the targeting vector. Where the selectable marker sequence is an antibiotic resistance gene, e.g., neo, the cells can be cultured in the presence of an otherwise lethal concentration of antibiotic, e.g., kanamycin. Those cells that survive have presumably integrated the targeting vector. If the selectable marker sequence is other than an antibiotic resistance gene, a Southern blot of the ES cell genomic DNA can be probed with a sequence of DNA designed to hybridize only to the marker sequence. If the selectable marker sequence is a gene that encodes an enzyme whose activity can be detected (e.g., beta-galactosidase or GFP), the enzyme substrate can be added to the cells under suitable conditions, and the enzymatic activity of the selectable marker sequence can be analyzed.

The targeting vector can integrate into several locations in the ES cell or germ cell genome, and can integrate into a different location in each cell's genome, due to the occurrence of random insertion events. The desired location of insertion is within a region of the ZCCHC6 endogenous gene sequence that eliminates functional ZCCHC6 protein expression. Typically, less than about 1 to about 10 percent of the cells that take up the targeting vector will actually integrate the targeting vector in the desired location. To identify those cells with proper integration of the targeting vector, chromosomal DNA can be extracted from the cells using standard methods known to those skilled in the art. The extracted DNA then can be probed on a Southern blot with a probe or probes designed selectively to hybridize to the targeting vector digested with (a) particular restriction enzyme(s). Alternatively, or additionally, a specific genomic DNA sequence can be amplified by PCR with probes specifically designed to amplify that DNA sequence such that only those cells containing the targeting vector in the proper position will generate DNA fragments of the proper size.

After suitable ES cells containing the targeting vector in the proper location have been identified, the transformed ES cells can be incorporated into an embryo. Incorporation can be accomplished in a variety of ways. A preferred method of incorporation of ES cells is by microinjection into an embryo that is at the blastocyst stage of development. For microinjection, typically, about 10-30 cells are collected into a micropipet and injected into a blastocyst to integrate the ES cell into the developing blastocyst.

The suitable stage of development for the blastocyst is species dependent, however for mice it is about 3.5 days. The blastocysts can be obtained by perfusing the uterus of pregnant females. Suitable methods for accomplishing this are known to the skilled artisan, e.g., as set forth by Bradley (in Robertson, ed., supra).

While any blastocyst of the right age/stage of development is suitable for use, preferred blastocysts are male and have genes coding for a coat color or other phenotypic marker that is different from the coat color or other phenotypic marker encoded by the targeted ES cell genes. In this way, the offspring can be screened easily for the presence of the targeting vector by looking for mosaic coat color or other phenotypic marker (indicating that the ES cell was incorporated into the developing embryo). Thus, for example, if the targeted ES cell line carries the genes for white fur, the embryo selected will preferably carry genes for black or brown fur.

After the ES cells have been incorporated, the transfected embryo can be implanted into the uterus of a pseudopregnant host. While any pseudopregnant host can be used, preferred hosts are typically selected for their ability to breed and reproduce well, and for their ability to care for their young. Such pseudopregnant hosts are typically prepared by mating with vasectomized males of the same species.

The pseudopregnant stage of the host mother is important for successful implantation, and it is species dependent. For mice, this stage is about 2-3 days pseudopregnant. As an alternative means to transfection of the targeting vector into an embryonic stem cell, the targeting vector can be transfected into an animal germ cell, i.e., an oocyte, e.g., a murine germ cell. Typically, retroviral vectors have been utilized to generate transgenic organisms by transfection of the viral vector into oocytes (Chan et al., Proc. Natl. Acad. Sci. USA 95:14028-33, 1998). Transgenic mice also were produced after the injection of exogenous DNA together with sperm heads into oocytes (Perry et al., Science 2841183, 1999).

It is contemplated by the present invention that transgenic animals can also be generated in vivo and in vitro (ex vivo), for example, by transfection, transduction, microparticle bombardment, or electroporation of vertebrate animal germ cells with the targeting vector together with a suitable transfecting agent. The in vivo method involves injection of the targeting vector directly into the testicle of the animal. In this method, all or some of the male germ cells within the testicle are genetically modified in situ, under effective conditions. The in vitro method involves obtaining germ cells from the gonad (i.e., testis) of a suitable donor or from the animal's own testis, using a novel isolation or selection method, transfecting or otherwise genetically altering them in vitro, and then returning them to the substantially depopulated testis of the donor or of a different recipient male vertebrate under suitable conditions where they will spontaneously repopulate the depopulated testis. The in vitro method has the advantage that the transfected germ cells can be screened by various means before being returned to the testis of the same or a different suitable recipient male to ensure that the transgene is incorporated into the genome in a stable state. Moreover, after screening and cell sorting only enriched populations of germ cells can be returned. These methods are more fully described in numerous references in the art, for example, PCT/US98/24238, which is incorporated herein by reference.

The male animal is then mated with a female animal of its species, and the progeny then are screened for transgenic animals.

Offspring that are born to the host mother can be screened initially for mosaic coat color or other phenotype marker where the phenotype selection strategy (such as coat color, as described above) has been employed. In addition, or as an alternative, chromosomal DNA obtained from tail tissue of the offspring can be screened for the presence of the targeting vector using Southern blots and/or PCR as described above and in Example below.

According to the present invention, the offspring that are positive for the ZCCHC6 targeting vector will typically be heterozygous, while homozygous disruption of ZCCHC6 gene is lethal. Naturally, the success of this approach requires that the technique employed yields polynucleotide products for detection that differ in length depending upon whether or not the targeting vector has been incorporated into the chromosomal copy of the ZCCHC6 locus. For example, if genomic analysis is performed using the Southern blot technique as described above, the restriction fragments predicted for endonuclease digestion of cells bearing the wild-type ZCCHC6 gene as opposed to cells bearing the recombinant ZCCHC6 genes must differ in length by an amount capable of being detected on an electrophoretic gel. This way, the transgenic animals that are heterozygous for incorporation of the targeting vector will yield two fragments of differing lengths that hybridize with the probe.

Those skilled in the art will readily appreciate that, although the mutation described herein has been inserted into the germ cells of a parent animal, e.g., mouse, the disrupted ZCCHC6 gene of the transgenic animal of the present invention ultimately will be present in the germ cells of future progeny and subsequent generations thereof. In addition, the genetic material is also present in cells of the progeny other than germ cells, i.e., somatic cells.

Other means of identifying and characterizing the ZCCHC6 heterozygous disruption mutant offspring are also available. For example, Northern blots can be used to probe mRNA obtained from various tissues of the offspring for the presence or absence of transcripts encoding either the mutated ZCCHC6 gene, the selectable marker sequence, or both. In addition, Western blots can be used to assess the level of expression of ZCCHC6 polypeptide product in various tissues of these offspring by probing the Western blot with an antibody against the ZCCHC6 protein, or an antibody against the selectable marker sequence protein product.

### Methods of Studying the Role of ZCCHC6 in Bone Disease or Development

Another aspect of the invention provides a method of studying the role of zinc-finger, CCHC domain-containing protein 6 (ZCCHC6) in bone disease or development. The method includes the steps of obtaining an animal model comprising a non-human animal having a heterozygous disruption of the expression of at least one endogenous gene encoding ZCCHC6, and determining differences in bone or cartilage characteristics, protein expression, or cytokine expression in the animal model in comparison to a corresponding wild-type non-human animal. The non-human animal model can be any of the non-human animal models described herein.

The method of studying the role of ZCCHC6 in bone disease or development includes determining differences in bone or cartilage characteristics, protein expression, or cytokine expression in the animal model in comparison to a corresponding wild-type non-human animal. Examples of bone characteristics include bone strength, bone mineral composition, bone cell composition (e.g., levels of osteoblasts, osteocytes, and osteoclasts), bone size, and bone density. Examples of cartilage characteristics include mechanical properties such as elasticity, frictional properties, thickness, and chemical composition (e.g., levels of proteoglycans or collagens). Methods of evaluating bone and cartilage characteristics are known to those skilled in the art.

A wide variety of proteins are involved in bone and cartilage biochemistry. Examples include osteocalcin, elastin, collagen, and bone morphogenic proteins. See Anusuya et al., J. Pharm Bioallied Sci. 8(Supp. 1), S39-S41 (2016) for a review of bone morphogenic proteins. A wide variety of cytokines are also involved in bone and cartilage biochemistry. Examples include bone morphogenic proteins (which are also considered cytokines), tumor necrosis factor-α, interleukin-1, interleukin-6, and interleukin-17. In some embodiments, the differences in protein expression or cytokine expression are evaluated in chondrocytes.

In some embodiments, the animal model is used to study bone disease. Bone disease, as used herein, also includes diseases that mainly affect cartilage. A wide variety of types of bone disease are known to those skilled in the art. Bone disease or disorders are characterized by a loss of bone mass and also encompass abnormalities in the strength and structures of the bones. Because of the role played by ZCCHC6 in inflammation, bone disease involving inflammation is of particular interest. Other types of bone disease are joint disease, and cartilage degenerative conditions. Examples of bone disorders include broken bones.

A wide variety of bone diseases and disorders are known to those skilled in the art. Examples of bone diseases and disorders include, but are not limited to: bone resorption, bone infection, osteoarthritis, osteoporosis, osteomalacia, osteitis fibrosa cystica, osteochondritis dissecans, osteomalacia, osteomyelitis, osteopenia, osteonecrosis, and porotic hyperostosis. Examples of broken bones include those resulting from a traumatic fracture, a critical sized bone defect, distraction osteogenesis, spine fusion surgery, joint replacement, an orthopaedic implant (bone implant). Examples of cartilage disease include osteoarthritis, achondroplasia, costochondritis, spinal disc herniation, and relapsing polychondritis.

In some embodiments, the bone disease being studied is osteoarthritis. Osteoarthritis (OA) is a disease which affects the whole joint and has a complex etiology with many factors that contribute to an increased risk of developing OA, including obesity, genetics, aging and trauma to the joint. When clinically evident, OA is characterized by joint pain, tenderness, limitation of movement, crepitus, occasional effusion, and variable degrees of inflammation without systemic effects. High levels of IL-6 and other pro-inflammatory cytokines (IL-1β, and TNF-α) are present in the synovial fluid of patients with OA and are also seen in animal models of OA. Kapoor et al., Nat Rev Rheumatol 7, 33-42 (2011). It is now known that in human chondrocytes (the only cell type present in cartilage) IL-1β stimulates the expression of several inflammatory mediators, including IL-6 which plays an important role in the pathogenesis of OA. Diagnosis of OA is typically based on signs and symptoms, with medical imaging using X-rays.

The ZCCHC6 knockout non-human animal model can also be used to study bone development. Long, short, and irregular bones develop by endochondral ossification, where cartilage is replaced by bone. Flat bones develop by intramembranous ossification, where bone develops within sheets of connective tissue. Compact cortical bone, representing about 80 percent of the mature skeleton, supports the body, and features extra thickness at the midpoint in long bones to prevent the bones from bending.

Bone growth is more complicated than simple elongation or simple enlargement. Most long bones add width on the outside by a process referred to as subperiosteal apposition (layers added to those already existing), while losing bone on the inside by endosteal resorption (breaking down and reabsorbing material at the center of a mass). At the same time, long bones gain in length by adding to the epiphyseal plate (the surface at the end of the bone). As they elongate, bones of this type go through a process called remodeling during which they change in outer shape as well. Conversely, the individual bones of the skull grow by circumferential apposition (adding layers at the circumference), while gaining in thickness by adding layers (apposition) at the surface with simultaneous resorption at the inner surface. For reviews of bone modeling, remodeling, and skeletal development, see Maggioli C. and Stagi S., Ann Pediatr Endocrinol Metab. 22(1), 1-5 (2017) and Berendsen A. and Olsen B., Bone, 80: 14-18 (2015).

Bone development can be studied at various different stages of bone development. In some embodiments, pre-natal bone development is studied. In other embodiments, to post-natal bone development is studied. Post-natal bone development includes early post-natal development from 0 to 2 years of age, regular post-natal development from 2 to 12 years of age, and bone development during puberty, which runs from 12 to 17 years of age (for human subjects).

In some embodiments, the method of studying bone disease and development can further include the step of administering ZCCHC6 protein (e.g., recombinant ZCCHC6 protein) to the animal model. By restoring normal levels of ZCCHC6 to the animal model, the effects of ZCCHC6 on bone or cartilage characteristics, protein expression, or cytokine expression can be further characterized.

Examples have been included to more clearly describe a particular embodiment of the invention and its associated cost and operational advantages. However, there are a wide variety of other embodiments within the scope of the present invention, which should not be limited to the particular examples provided herein.

### EXAMPLES

### Example 1: Mice Lacking TUT7 (Zcchc6) have High Bone Mass Associated with Increased Osteoblasts Differentiation

In this study, we investigated the role of TUT7 in skeletal development and remodeling. We generated TUT7 knockout (KO) mice and characterized their skeletal phenotype. We discovered that deletion of TUT7 was not lethal and neonatal TUT7KO mice developed normally similar to wildtype (WT) littermates; however, postnatally TUT7KO mice have significantly increased bone mass compared to WT. Ex vivo analysis of bone marrow-derived osteoprogenitor cells demonstrated an increase in osteoblast differentiation and mineralization suggesting that TUT7 plays an important role in OB function. Interestingly, osteoclasts derived from TUT7KO mice ex vivo showed no significant difference in differentiation and function. Gene expression analysis using a PCR-based array showed that constitutive expression of master transcription factor Osterix (Osx) was significantly upregulated in OBs derived from TUT7KO mice compared to the expression levels in OBs from WT littermates. Furthermore, TUT7 overexpression in WT osteoblasts resulted in suppression of Osx expression and activity while absence of TUT7 resulted in enhanced Osx expression and activity. Overall, our data suggests that TUT7 is a negative regulator of bone formation through suppression of Osx in OBs. Taken together, this study is the first to demonstrate the role of TUT7 in postnatal skeletal development and remodeling.

### Materials and Methods

Cell Culture and Reagents.

Minimum essential medium alpha (α-MEM) was purchased from Mediatech (Manassas, Va.). The preosteoblast-like cell line MC3T3-E1 subclone 4 was from ATCC (Manassas, Va.). Alcian Blue, Alizarin Red, Silver Nitrate, Sodium Thiosulfate, Fast Green, Sodium Tartrate, Sodium Acetate, Trypsin, Penicillin-Streptomycin, Amphotericin-B and Ascorbic Acid were from Thermo Fisher Scientific (Waltham, Mass.), β-glycerophosphate, Dexamethasone, Sodium Carbonate, Collagenase B and Fast Red Violet were from Sigma-Aldrich (St. Louis, Mo.). Toluidine blue was from Electron Microscopy Sciences (Hatfield, Pa.). The RANKL and M-CSF were from R&D systems (Minneapolis, Minn.), Osterix antibody was from Abcam (Cambridge, UK), GAPDH antibody was from Cell Signaling (Danvers, Mass.), and TUT7 antibody and Calcein were from Santa Cruz Biotechnology (Dallas, Tex.). PCR Primers were purchased from IDT (Coralville, Iowa). The pcDNA3 expression vector was purchased from Invitrogen (Carlsbad, Calif.). pcDNA3-FLAG-mTUT7 was a gift from Zissimos Mourelatos (Addgene Plasmid #60044). pGL3-Basic luciferase vector was purchased from Promega (Madison, Wis.). The pGL3-osterix luciferase reporter construct was a kind gift from Dr. Mark Nanes (Emory University).

Generation of TUT7KO Mice.

Mouse embryonic stem (ES) cell line (AEO325) containing a gene-trap insertion in the Zcchc6 gene (MMRRC/KOMP, University of California-Davis) was used to produce heterozygous Zcchc6 KO mice. The gene-trap genomic insertion site was located within introns 2-6. All known conserved protein motifs and domains in Zcchc6/TUT7 are downstream of the insertion site. Appropriate targeting by 5′ and 3′ homology arms was confirmed by PCR. Rapid amplification of C-DNA ends (RACE) demonstrated the presence of Exons 2-6 of Zcchc6 (SEQ ID NO: 3) in the ES cells. TUT7KO mice were generated by crossing heterozygous mice and displayed normal Mendelian ratio.

Skeletal Preparations.

Whole skeletal preparations from day 5 newborn mice were prepared essentially as previously described. Wassersug R J, Stain Technol 51(2):131-134 (1976) Images were taken using a Nikon SMZ 800 stereoscope (Nikon, Melville, N.Y.).

Micro-CT Analysis.

Femurs from 8- and 16-weeks old male and female WT and TUT7KO mice (n≥5) were analyzed using a SkyScan 1172 high-resolution microtomography (MicroCT) system (Bruker, Billerica, Mass.) as previously described. Abdelmagid et al., The American journal of pathology 184(3):697-713 (2014) Scanned images were reconstructed using the NRecon software. Following reconstruction, samples were analyzed using the CTAn software. The trabecular regions of interest were taken 400 μm below the growth plate and extended 5,700-6,000 μm depending on age proximally towards the diaphysis. Percentage of bone volume per tissue volume (BV/TV; %), trabecular number (Tb.N; no./mm), trabecular separation (Tb.Sp.; μm), and trabecular thickness (Tb.Th; μm) were measured and analyzed using the SkyScan CT analyzer software. Three-dimensional reconstructed images of the sagittal and axial planes of the femoral metaphysis were generated using the SkyScan CTvox software (Skyscan).

Dual-energy X-Ray Absorptiometry (DEXA) and Imaging.

Mouse whole body and femur bone mineral density (BMD; g/cm²) were analyzed using the Lunar PIXImus densitometer (GE Medical Systems, Madison, Wis.). Freshly euthanized WT and TUT7KO whole mice or femurs were placed on a specimen tray and scanned. For each specimen, the region of interest was selected and analyzed. Skeletal x-ray images of WT and TUT7KO were acquired using the IVIS Lumina XRMS Series III (PerkinElmer, Waltham, Mass.).

ELISAs.

Plasma samples were collected from 8- and 16-weeks old WT and TUT7KO male and female mice (n≥5) by cardiac puncture. WT and TUT7KO plasma were analyzed by enzyme-linked immunosorbent assays (ELISA) to determine the levels of Osteocalcin (Biomedical Technologies, Stoughton, Mass.), CTX-I (MyBiosource, San Diego, Calif.), RANKL and OPG (R&D Systems) according to the manufacturer's instructions. To obtain the RANKL/OPG ratio for each animal, the level of RANKL was divided by the OPG level from the same animal and standardized based on WT levels.

Histology and Bone Histomorphometric Analysis.

For histological analyses of osteoblasts and mineralized bone matrix, distal femurs from 8-weeks old male and female mice (n≥4) were dissected and fixed in 4% formaldehyde, dehydrated, and embedded undecalcified in methylmethacrylate resin. Sagittal sections were cut at 5 μm using a microtome and carbide knife. Sections were then stained with von Kossa and counterstained with 2% Toluidine Blue as previously described. Frara, et al. Journal of cellular physiology 231(1):72-83 (2016) For histological analyses of osteoclasts, distal femurs from 16-weeks old male and female mice were fixed, decalcified, paraffin embedded, and cut into 5 μm sections. Sections were stained with Tartrate Resistant Acid Phosphatase (TRAP) and counterstained with 0.02% Fast Green in order to visualize osteoclasts on the bone surface. Bright field images were acquired using a Nikon Ti Eclipse inverted microscope (Nikon).

Quantitative histomorphometry was performed using the Osteomeasure software version 3.2.1 (Osteometrics, Decatur, Ga.). Images were acquired using a bright field microscope at 10× and 20× magnification equipped with a digital color video camera (Olympus, Center Valley, Pa.). Analyses were performed in an area 100-600 μm proximal to the growth plate. Three-dimensional parameters included trabecular number (Tb.N; No./mm) and trabecular separation (Tb.Sp; μm). Two-dimensional parameters included osteoblast number per bone perimeter (N.Ob/B.Pm; no./mm), percentage of osteoblast surface per bone surface (Ob.S/BS; %), osteoclast number per bone perimeter (N.Oc/B.Pm; no./mm), and percentage of osteoclast surface per bone surface (Oc.S/BS; %).

For dynamic histomorphometry, 8-weeks old WT and TUT7KO mice were injected subcutaneously (10 mg/kg) with Calcein AM 7 and 2 days before sacrifice. Femurs were collected, fixed, dehydrated, and embedded undecalcified in methylmethacrylate. Sections were cut, imaged and analyzed using a Nikon Eclipse Ti inverted microscope. Single-labeled surface, double layered surface, mineralizing surface, mineral apposition rate (MAR), and bone formation rate (BFR) were calculated as previously described (10).

Isolation and Analyses of Bone Marrow-Derived and Calvaria-Derived Osteoblast Cultures.

Bone marrow progenitor cells from the long bones of 8-weeks old WT and TUT7KO mice were flushed and cultured in α-MEM containing 10% fetal bovine serum (FBS), 1% penicillin-streptomycin (PS), and 0.1% amphotericin-B (Amp-B). Calvarial primary osteoblasts from WT and TUT7KO five-day old pups were isolated and digested with 0.25% Trypsin and 0.1% Collagenase B.

For osteoblast differentiation, bone marrow progenitor cells or primary osteoblasts were cultured in 10% FBS, 10 mM β-glycerophosphate, 50 μg/mL ascorbic acid, and 10⁻⁷ M dexamethasone. Osteoblast matrix maturation was assessed by alkaline phosphatase (ALP) staining and activity on undifferentiated (day 0) and differentiated (day 7) cultures using a kit (Sigma-Aldrich; Anaspec, Fremont, Calif.).

Osteoblast matrix mineralization was assessed by von Kossa staining. Briefly, bone marrow progenitor cells or primary osteoblasts were differentiated with osteogenic media for 21 days and fixed in 10% formalin. Fixed cultures were then stained with 5% silver nitrate and washed with dH₂O. Following washing, sodium carbonate and sodium thiosulfate were added to visualize mineralized nodules. Images for each well were analyzed using the NIS-Elements software.

Isolation and Analysis of Bone Marrow-Derived Osteoclast Cultures.

Bone marrow progenitor cells from 8-weeks old WT and TUT7KO mice were obtained as previously described. Abdelmagid S M, et al., The Journal of biological chemistry 290(33):20128-20146 (2015) Mature osteoclasts were analyzed by TRAP activity assay. Briefly, mature osteoclasts plated in 96-well plates were fixed with 10% formalin and washed with dH₂O. For TRAP activity assays, a 1:1 ratio of methanol:acetone was added to cultures followed by incubation with TRAP buffer (52 nM of Na+Tartrate in 0.1 M Na+-Acetate buffer) containing 0.1 mg/mL of p-nitrophenyl phosphate (p-NPP) (Thermo Fisher) for 1 hour at 37° C. Following incubation, 1 N NaOH was added to cultures and the optical density was read using a Synergy H4 microplate reader.

For TRAP staining, mature osteoclasts were incubated with TRAP buffer containing 1.5 mM Napthol-AS-MX phosphate and 0.5 mM Fast Red Violet. TRAP positive osteoclasts (n≥3) were counted and imaged using NIS-Elements software.

Osteoclast-mediated resorption was assessed by plating WT and TUT7KO bone marrow progenitor cells on Corning® OsteoAssay surfaces (Corning, Corning, N.Y.) and differentiated with M-CSF and RANKL as described above. Upon generation of mature osteoclasts, cultures were terminated using 10% bleach. Resorption areas were quantitated using NIS-Elements software.

Analysis of Osteoblast-Mediated Osteoclastogenesis Using Co-Culture Assay.

Primary calvarial osteoblasts from WT and TUT7KO were plated at 4.0×10⁴ cells/cm² in 48-well plates and treated with 1,25-dihydroxyvitamin D (10⁻⁸M) and prostaglandin E2 (PGE2; 10⁻⁶M) (Sigma-Aldrich). The next day, bone marrow non-adherent cells were co-cultured with osteoblasts at a density of 3.0×10⁵ cells/cm². Mature osteoclasts were evident within 7-10 days and were assessed by TRAP activity, staining, and count as described above.

Western Blotting.

Cells were lysed in RIPA buffer and the total lysate protein was quantified using a BCA protein assay kit (Thermo), resolved by SDS-PAGE and transferred to a polyvinylidene fluoride (PVDF) membrane (BioRad, Hercules, Calif.). Membranes were probed overnight with primary antibodies against TUT7, Osterix, GAPDH, followed by incubation with the appropriate secondary HRP conjugated antibodies and the immunoreactive bands were visualized using chemiluminescent substrate (Millipore, Billerica, Mass.) and imaged on Syngene PXi system (Syngene, Rockville, Md.). Densitometric analysis was performed using the Syngene software.

Quantitative Real-Time PCR (RT-qPCR).

Total RNA was isolated from WT and TUT7KO samples from tissue or cells as previously described. Sondag et al., Journal of cellular physiology 229(7):955-966 (2014) Following RNA isolation, cDNA was prepared using a High Capacity cDNA Reverse Transcription kit (Life Technologies). Quantitative (q) RT-PCR was performed with the Step-one qPCR system with the 2×SYBR Green PCR Master Mix (Applied Biosystems, Foster City, Calif.) and relative mRNA expression of osteoclast-related genes was determined using the ▴▴C_{T} method with GAPDH as an internal control.

Osteogenesis Gene Expression Array.

Total RNA was isolated from WT and TUT7KO osteoblasts using Qiazol and purified using an RNA extraction kit (Qiagen). Gene expression profiling was performed using a mouse osteogenesis RT² Profiler PCR array (Qiagen) and StepOnePlus system (Applied Biosystems). Relative gene expression was analyzed using the SABiosciences PCR array data analysis web portal.

Transfection and Luciferase Activity Assay.

WT and TUT7KO osteoblasts or MC3T3-E1 osteoblast like cells were transfected using FuGene® HD transfection reagent (Promega). Cells were transfected or co-transfected with either PGL3 basic, PGL3-OSX, pcDNA3, pcDNA3-FLAG-TUT7 using FuGene® HD transfection reagent. Forty-eight hour post-transfection, cells were harvested and assayed using the Dual Luciferase assay system (Promega). The luciferase activity values were normalized based on Renilla values to correct for variation in transfection efficiency.

Data and Statistical Analyses.

For all data generated, differences between individual groups were analyzed using Prism software version 5.04 (GraphPad, La Jolla, Calif.). All experiments were repeated 3-5 times with similar results. In cases involving the comparison of two groups, an unpaired t-test was performed. In cases when multiple groups were being compared, a one-way analysis of variance (1-way ANOVA) was employed along with Tukey's multiple comparison post hoc test. Group means or means±standard error of the mean (±SEM) was graphed. All differences where p<0.05 were regarded as statistically significant.

### Results

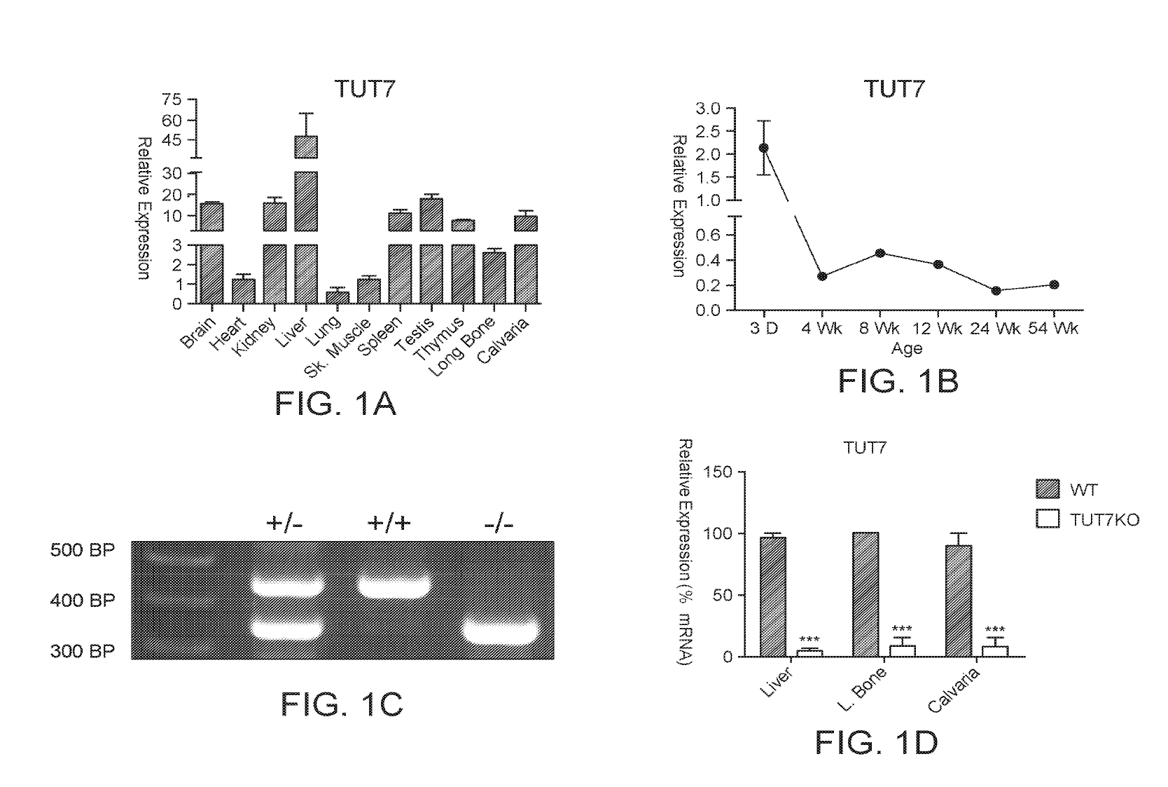

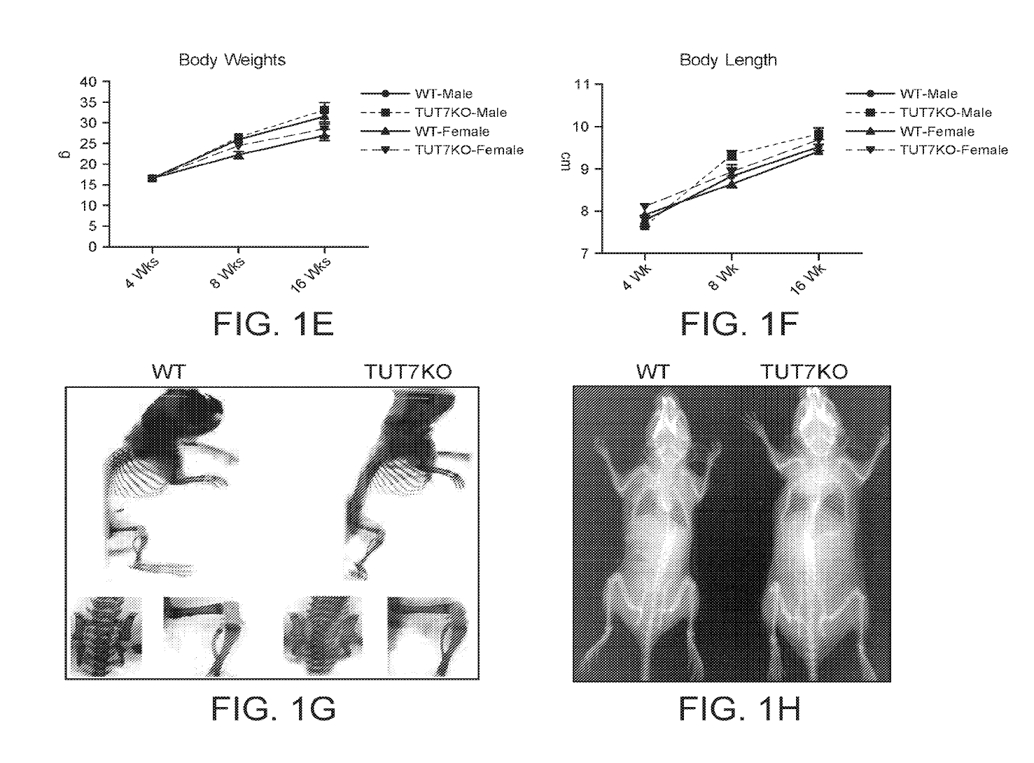

TUT7 is differentially expressed with age. We first quantified the expression of TUT7 in bone and soft tissues of mice by qPCR. Our data showed that TUT7 mRNA was highly expressed in liver, brain and kidney of 8-weeks old mice (FIG. 1A). Interestingly, TUT7 mRNA was also highly expressed in calvaria and long bones. Next, we determined the expression of TUT7 in aging bone and discovered that the expression of TUT7 in bone was highest at day 3 but was drastically reduced by 4 weeks of age and then remained low throughout ontogeny (FIG. 1B). These findings indicated that TUT7 may play an important role in early postnatal development of the skeletal system.

TUT7 deficient mice develop normally. To determine the role of TUT7 in skeletal development and remodeling we derived a line of TUT7/Zcchc6 gene mutant mice and used PCR to distinguish homozygous (KO, −/−), heterozygous (Het, +/−), and wildtype (WT, +/+) genotypes (FIG. 1C). In addition, we confirmed the absence of TUT7 mRNA expression in multiple tissues of the KO mice by qPCR (FIG. 1D). TUT7KO mice developed normally and absence of the gene did not have a significant effect on either body weight (FIG. 1E) or body length (FIG. 1F). Furthermore, there did not seem to be any dramatic or noticeable differences in morphology or skeletal structure during early development (FIG. 1G) or adult stage (FIG. 1H).

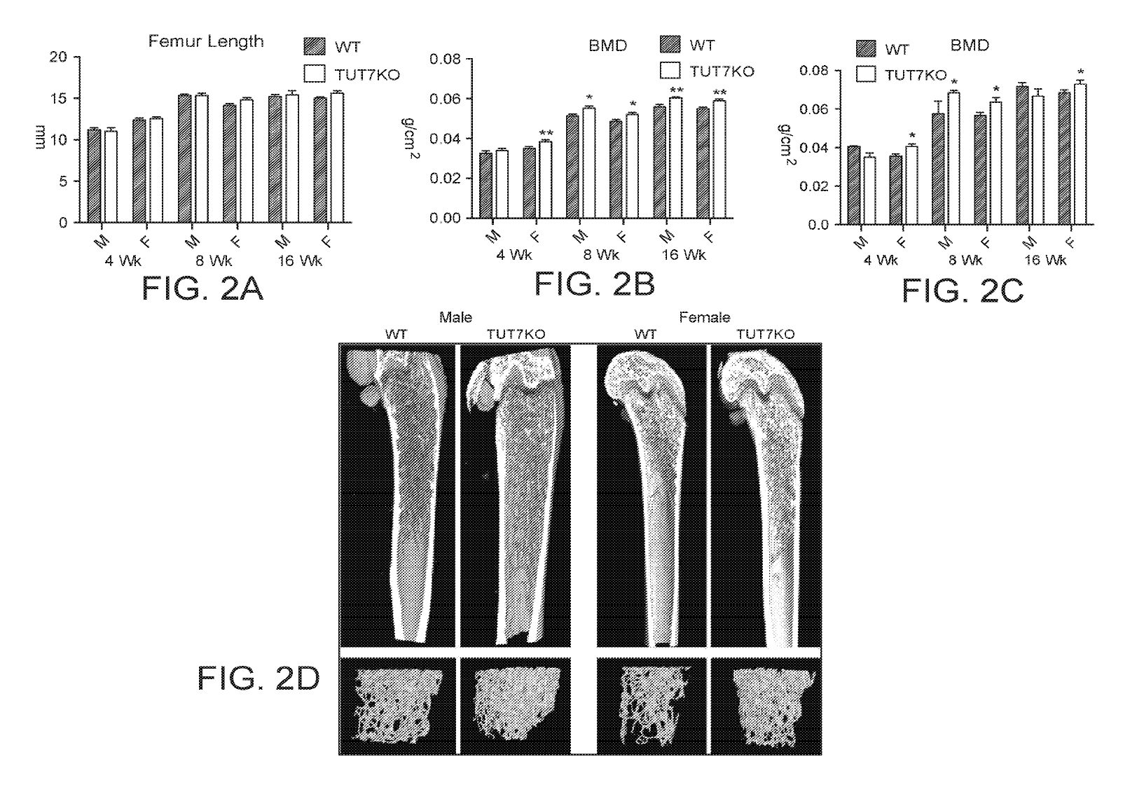

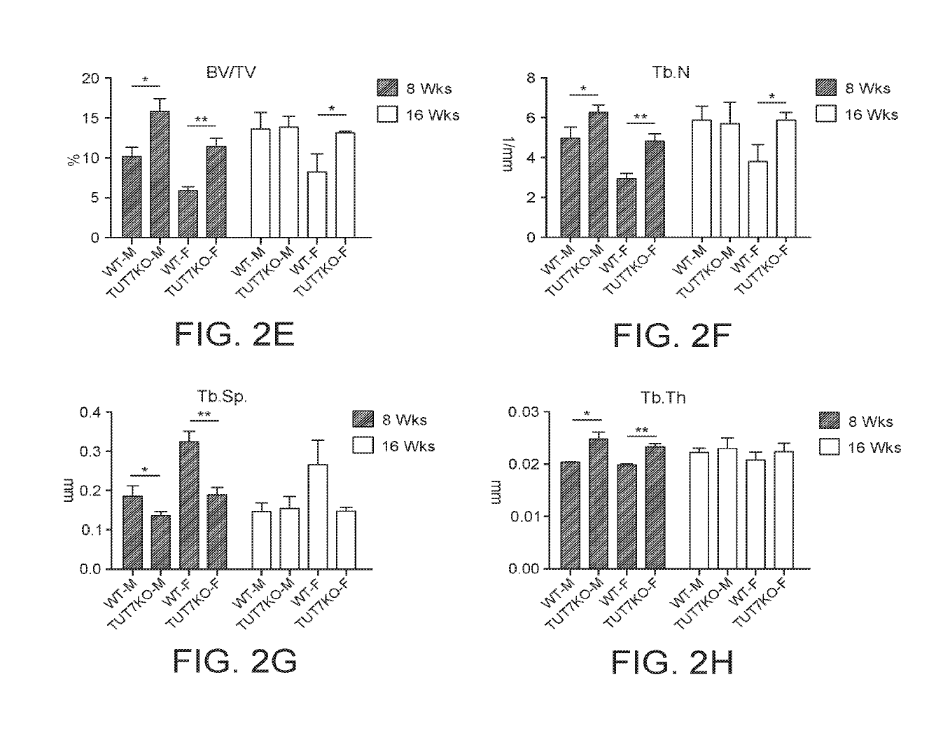

Absence of TUT7 enhances bone mass in vivo. Analyses of the bones of TUT7KO mice showed no difference in femur length but TUT7KO mice had increased whole body (FIG. 2B) and femoral BMD compared to WT mice (FIG. 2C). Interestingly, TUT7KO mice femurs showed a dramatic increase in the trabecular bone mass compared to WT mice (FIG. 2D). Further analyses revealed a significant increase in BV/TV, Tb.N, Tb. Sp., and Tb.Th in 8 weeks old TUT7KO mice compared to WT mice (FIG. 2E-H). Interestingly, the increase in bone mass was greater in TUT7KO female mice than in males. Furthermore, microCT analysis of femurs from 16-weeks old mice revealed a significant increase in BV/TV and Tb.N in TUT7KO female mice but not in male mice (FIG. 2E-H). This indicated that TUT7 may have an age-dependent and gender-specific role in the regulation of bone mass, for reasons not clear at present.

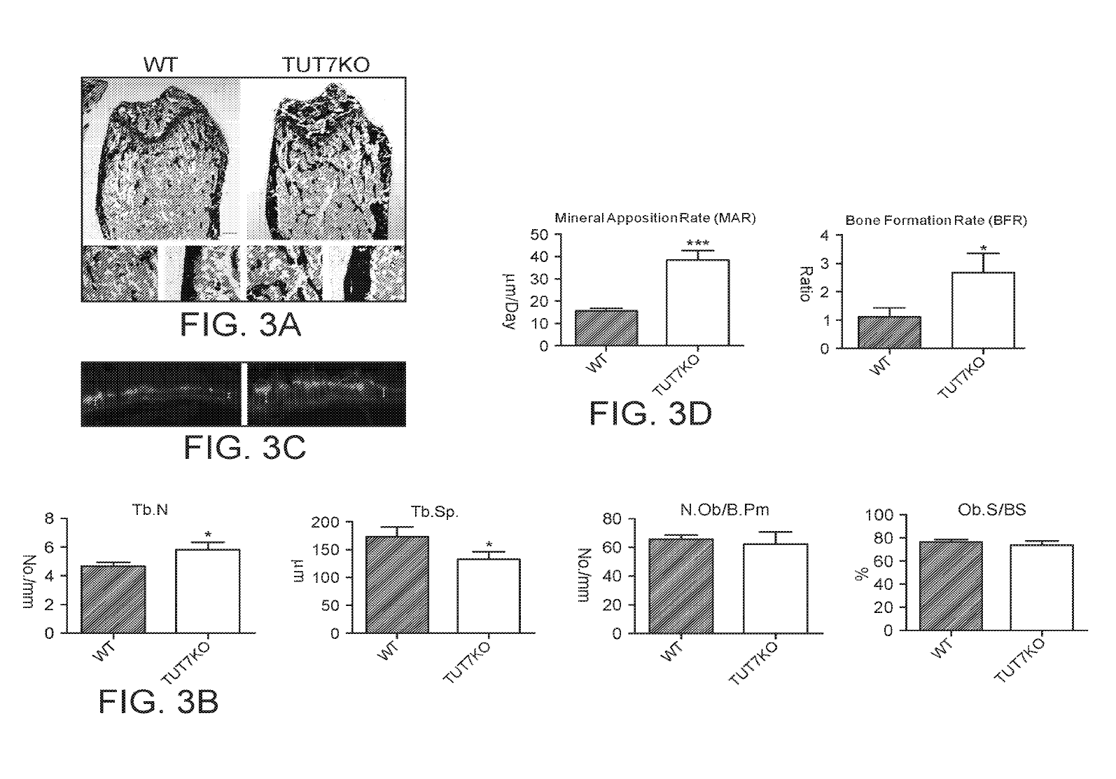

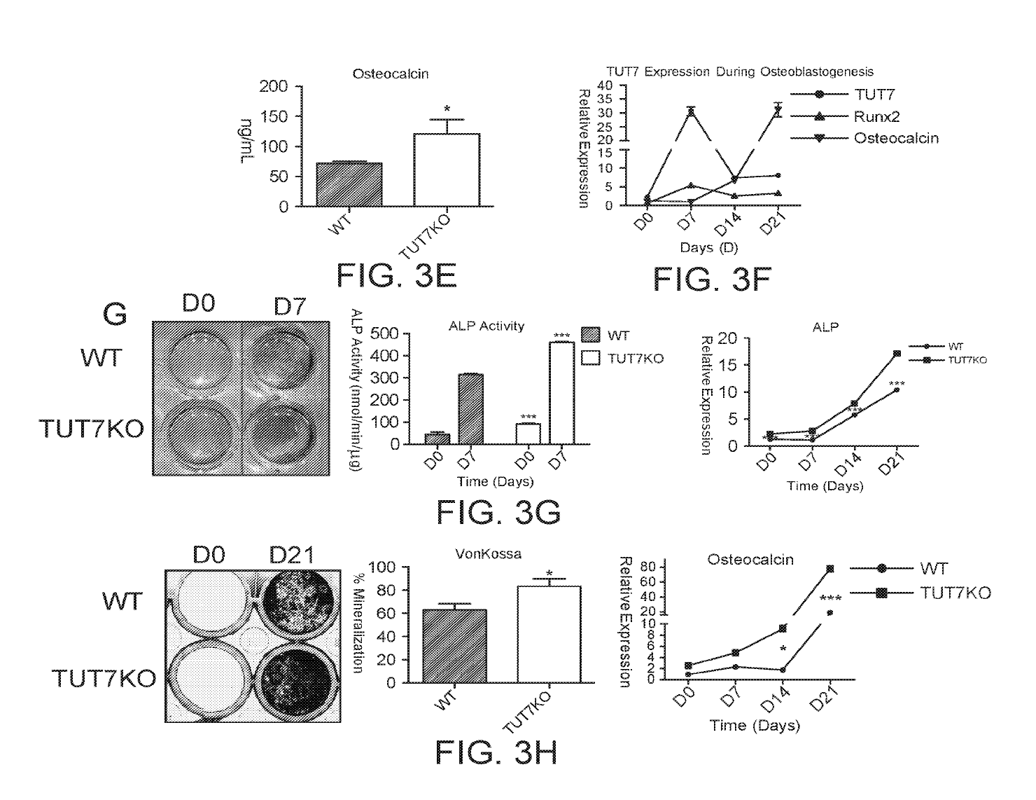

Deficiency of TUT7 enhances bone formation in vivo and increases osteoblast differentiation ex vivo. To understand the cause of increase in bone mass in TUT7KO mice, we examined bone formation in vivo by histomorphometric analyses. Femurs from 8-weeks old WT and TUT7KO mice were stained for von Kossa and analyzed. Femurs from TUT7KO mice showed a significant increase in trabecular number (Tb.N) and trabecular spacing (Tb.Sp.) (FIG. 3A-B). Interestingly, there seemed to be no significant difference in the number of osteoblasts (N.Ob/B.Pm) or osteoblast bone surface (Ob.S/BS) in these mice. This indicated that the increase in bone matrix mineralization may be due to an increased activity of the osteoblasts rather than an increase in numbers in the TUT7KO mice. To test this, we injected WT and TUT7KO mice with Calcein to assess the dynamic mineralization rate in vivo. Our results showed that TUT7KO mice had ˜150% increase in mineral apposition rate (MAR) and bone formation rate (BFR) compared to WT mice (FIG. 3C-D). Furthermore, TUT7KO mice showed a significant increase (p<0.05) in the serum levels of osteoblast marker osteocalcin compared to the levels in wild type mice (FIG. 3E). These results demonstrated that absence of TUT7 enhances bone formation and mineralization in vivo.

Next, we examined the TUT7 expression during osteoblastogenesis, and determined its role in osteoblast differentiation. Interestingly, TUT7 expression was greatly enhanced at day 7 of osteoblast differentiation, but decreased thereafter (FIG. 3F). Osteoblasts derived from TUT7KO mice did not expressed TUT7 but showed a significant increase in ALP staining, activity, and mRNA expression compared to osteoblasts derived from WT mice (FIG. 3G). Expression of ALP was significantly high in TUT7KO cultures compared to WT even without osteogenic media treatment (DO) indicating that endogenously, TUT7 may regulate specific factors related to osteoblast commitment. Next, we assessed the role of TUT7 in late stage osteoblast differentiation and matrix mineralization. Osteoblast cultures from TUT7KO mice showed a significant increase in osteoblast matrix mineralization and osteocalcin expression compared to cultures derived from WT mice (FIG. 3H). These results demonstrated that absence of TUT7 has a significant impact on osteoblast differentiation and matrix mineralization in vivo.

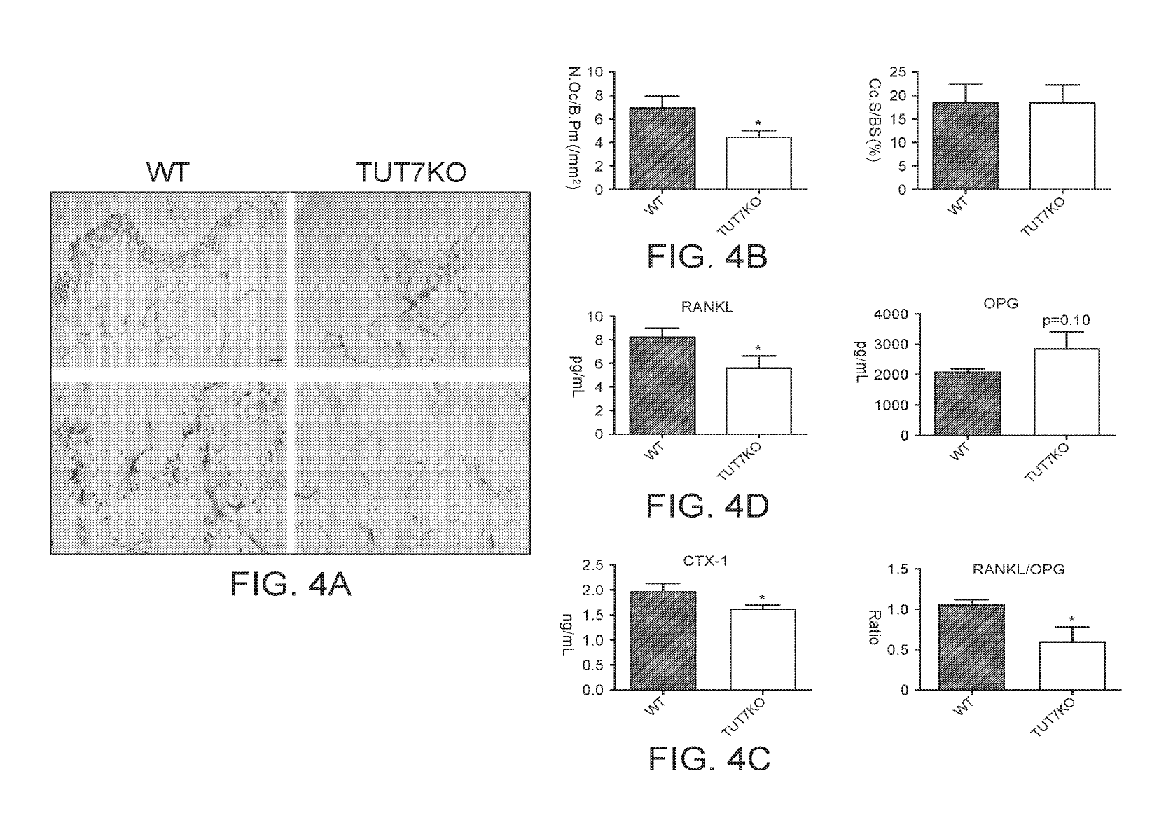

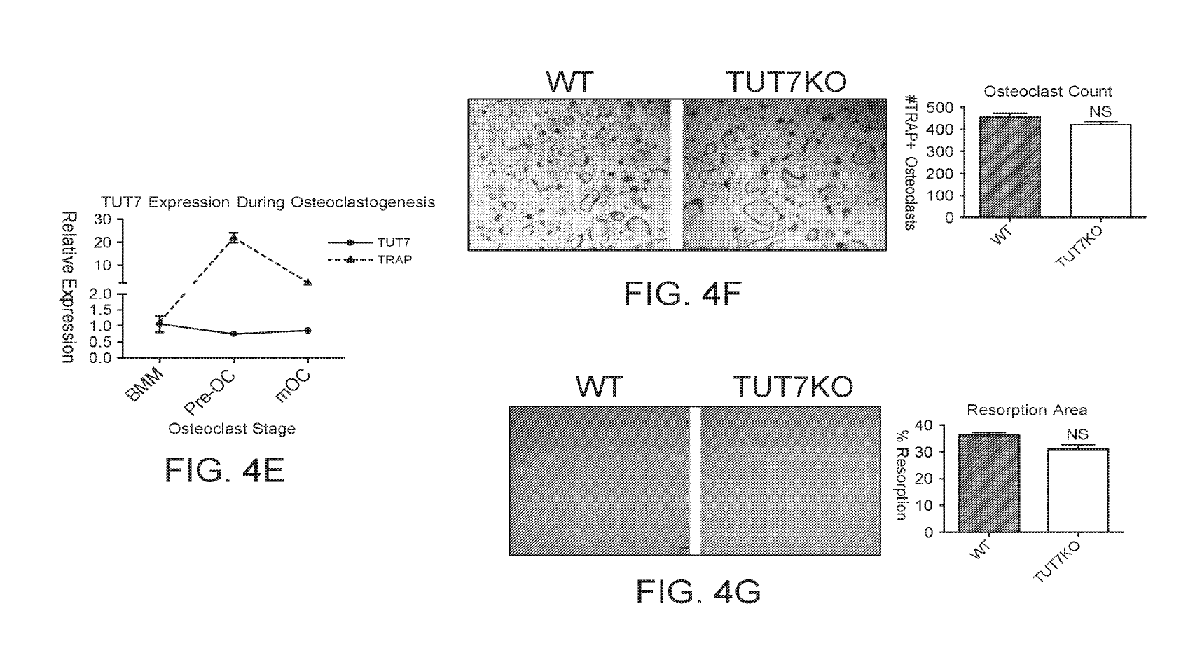

Absence of TUT7 inhibits osteoclasts in vivo, but not ex vivo. Next, we evaluated the role of TUT7 in osteoclastogenesis to determine if this may contribute to the enhanced bone mass observed in the TUT7KO mice. TRAP staining and histomorphometric analyses in the TUT7KO mice revealed a significant reduction in the number of osteoclasts (N.Oc/B.Pm), but no difference in the osteoclast surface (Oc.S/BS) compared to WT mice (FIG. 4A-B). This indicated that the absence of TUT7 affect the number of mature osteoclasts but not their size or morphology in vivo. Serum levels of CTX-1 and RANKL were significantly reduced in the TUT7KO mice but no significant difference in the levels of OPG was detected; however, there was a reduction in the overall RANKL/OPG ratio in the TUT7KO mice compared to WT mice (FIG. 4C-D). These results indicated that TUT7 may play a role in the differentiation and activity of osteoclasts in vivo.

Since the absence of TUT7 suppressed the generation of mature osteoclasts in vivo, we evaluated the role of TUT7 during osteoclastogenesis ex vivo. Expression of TUT7 mRNA during osteoclastogenesis in vitro was not altered during osteoclast differentiation from WT mice (FIG. 4E). Additionally, when bone marrow progenitor cells from WT and TUT7KO mice were differentiated towards osteoclasts, there was no significant difference in osteoclast differentiation as determined by TRAP staining and count or osteoclast resorption (FIG. 4F-G). Taken together our data indicated that the absence of TUT7 affected osteoclast differentiation and function in vivo but not ex vivo suggesting the involvement of other factors in osteoclast differentiation and function in vivo that may be regulated by TUT7.

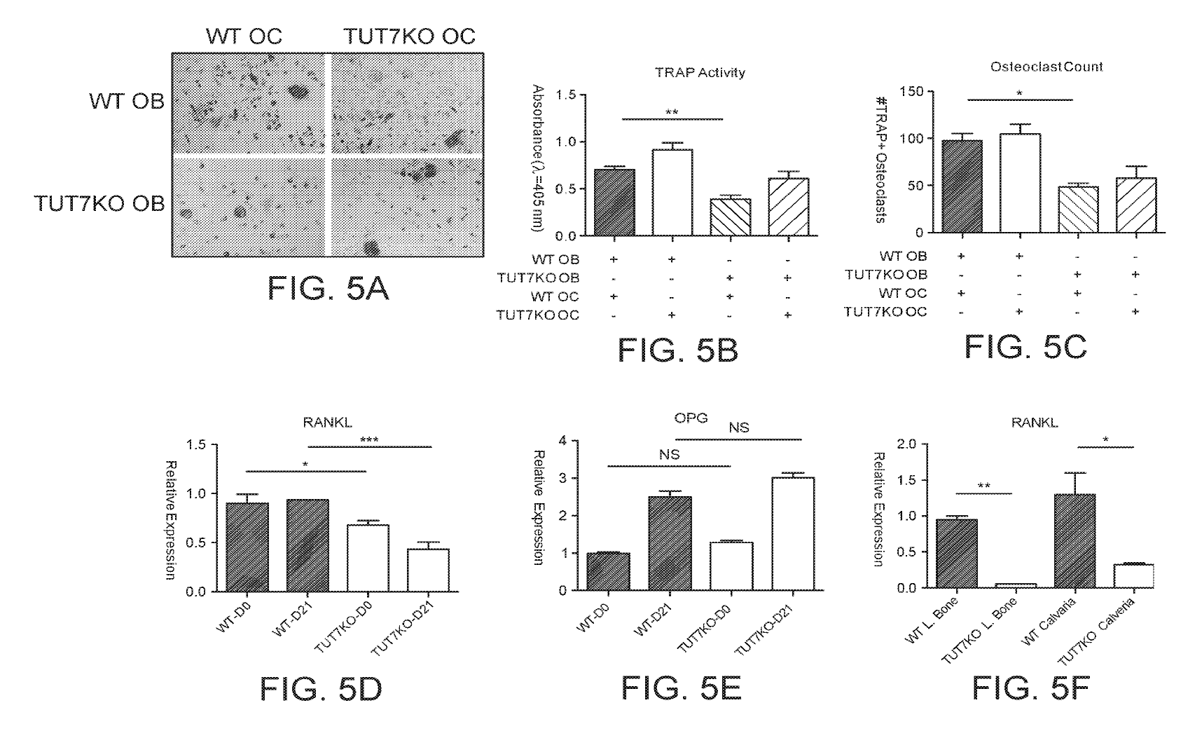

Absence of TUT7 regulates osteoblast-mediated osteoclastogenesis. To address the discrepancy observed in TUT7KO osteoclasts in vivo and ex vivo, we used an osteoblast and osteoclast “mix and match” co-culture system. There was no difference in osteoclast differentiation in WT osteoblasts (OB) co-cultured with WT osteoclasts (OC) compared to WT OB co-cultured with TUT7KO OCs. Importantly, there was a significant reduction in osteoclast differentiation when TUT7KO OBs were co-cultured with WT OCs compared to WT OBs co-cultured with WT OCs as shown by TRAP staining, activity, and count (FIG. 5A-C). Furthermore, the expression of RANKL, but not OPG, was significantly downregulated in TUT7KO osteoblasts compared to WT osteoblasts (FIGS. 5D-E) and also in long bones and calvaria of TUT7KO mice compared to WT mice (FIG. 5F). These results indicated that the role of TUT7 in osteoclast differentiation is indirectly mediated through regulation of RANKL expression in osteoblasts.

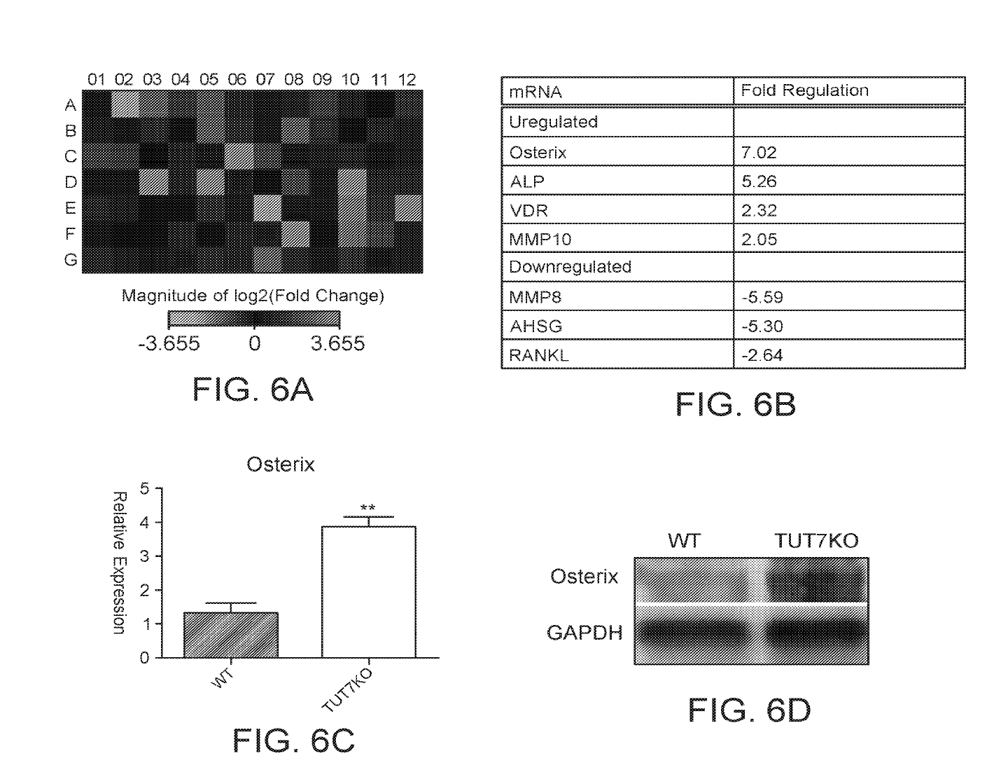

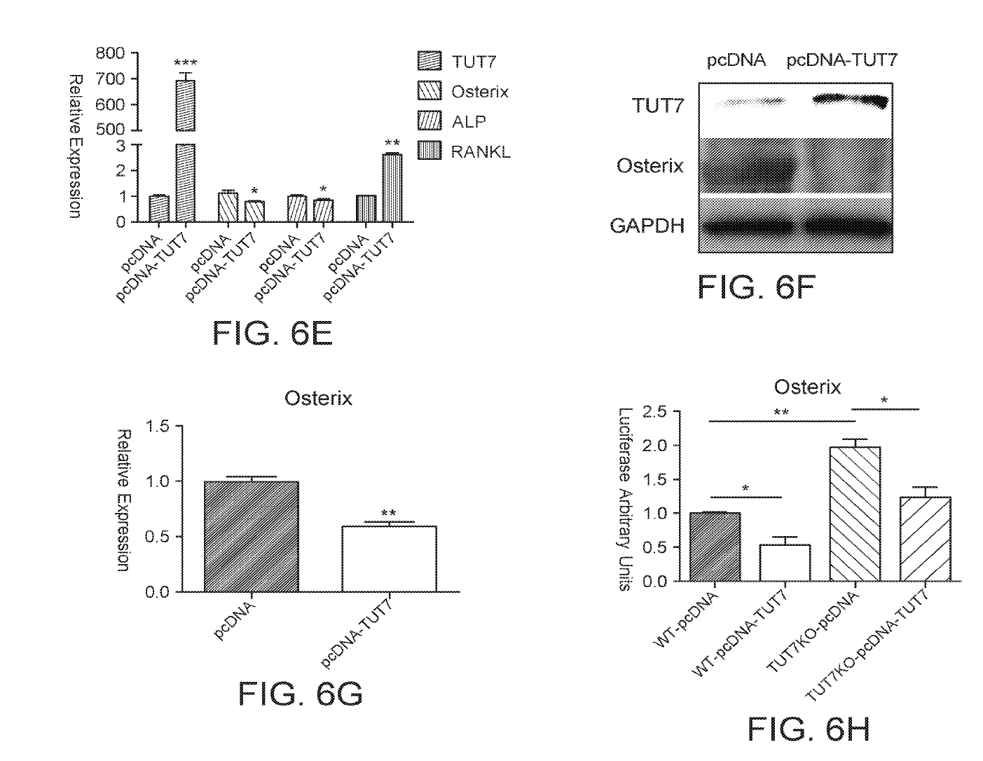

TUT7 regulates the expression of the master transcription factor Osterix in osteoblasts. We also analyzed osteoblast-related genes expression using a PCR-based osteogenesis gene array and identified a number of dysregulated genes including a seven-fold increase in the expression of the transcription factor Osterix mRNA (FIG. 6A-B). The upregulation of Osterix expression was confirmed at both the mRNA and protein levels (FIG. 6C-D) indicating that TUT7 is a negative regulator of Osterix expression. Importantly we found that overexpression of TUT7 decreased the expression of Osterix and ALP mRNAs, and increased RANKL mRNA expression (FIG. 6E). Furthermore, MC3T3-E1 osteoblast-like cells overexpressing TUT7 gene, had significantly reduced levels of Osterix protein and transcriptional activity (FIG. 6F-G). We also overexpressed TUT7 gene in WT and TUT7KO osteoblasts and examined the transcriptional activity of Osterix by luciferase activity assay (FIG. 6H). Significantly high levels of Osterix transcriptional activity was observed in the TUT7KO osteoblasts but was reduced in WT cells overexpressing TUT7 as expected. Interestingly, when TUT7 was ectopically expressed in osteoblasts derived from TUT7KO mice, Osterix activity was restored to WT control levels. Overall, this data demonstrated that TUT7 negatively regulates Osterix expression and activity in osteoblasts.

### DISCUSSION

In the present study, we investigated the role of TUT7/Zcchc6 in bone remodeling in mice in vivo. We discovered that absence of TUT7 has no effect on the mice's viability, reproduction, or development. Interestingly, other studies of canonical and non-canonical PAPs mutants have also described a similar phenotype including the report that PAP GLD2/TUT2 knockout (TUT2KO) mice developed normally. Nakanishi et al., Biochemical and biophysical research communications 364(1):14-19 (2007) Furthermore, absence of Zcchc16/Sirh11 also did not affect development; however, Zcchc16KO mice were shown to have dysfunctional cognitive abilities. Inci et al., PLoS genetics 11(9):e1005521 (2015) Interestingly, in the TUT4/Zcchc11KO mice, 50% of mutant neonates died and the remaining mice displayed poor growth and development indicating its importance in growth and skeletal development. Koga et al., Nature medicine 11(8):880-885 (2005) However, such studies with TUT7/Zcchc6 have not been reported.

We found that TUT7 was ubiquitously expressed in different tissues including bone. We discovered that absence of TUT7 results in enhanced bone mass in vivo, a finding not previously reported for any of the known members of the TUT family. Although knockout of TUT7 expression had a significant effect on the skeletal phenotype in both sexes, but the absence of TUT7 expression had a greater impact on females than males. Skeletal sexual dimorphism can generally be attributed to alterations in sex steroids and growth hormones including estradiol, growth hormone, and IGF. Callewaert et al., The Journal of endocrinology 207(2):127-134 (2010) Previous studies have shown that mice with mutation in high density lipoprotein (HDL) scavenger receptor class B type I (SR-BI) have enhanced bone mass in females but not in males due to alterations in leptin levels. Martineau et al., The Journal of endocrinology 222(2):277-288 (2014) Interestingly, gender-related differences in regards to the effects of leptin on bone have been documented. Thomas et al., Bone 29(2):114-120 (2001) Future studies should determine if TUT7 plays a role in endocrine regulation of bone homeostasis.

Another important finding was that the absence of TUT7 expression decreases osteoclast differentiation and bone resorption in vivo, but not ex vivo. This phenomenon can be partially explained due to a decrease in the levels of RANKL as seen in plasma, bone, and osteoblast cultures of the TUT7KO mice. A previous report has shown that Zcchc11/TUT4 negatively regulates IL-6 production through the uridylation of miR-26a. Jones et al., Nature cell biology 11(9):1157-1163 (2009) Another study has shown that Zcchc11/TUT4 negatively regulates LPS-induced NFκB activation. Minoda et al., Biochemical and biophysical research communications 344(3):1023-1030 (2006) Furthermore, Zcchc9 was shown to suppress transcriptional activation of NFκB and serum response element (SRE) and may have a role in regulating MAPK signaling. Zhou et al., Journal of genetics and genomics, 35(8):467-472 (2008) This indicates that members of the Zcchc/TUT enzyme family may regulate inflammatory cytokine expression and intracellular signaling. Activation of NFκB has been shown to inhibit osteoblast differentiation (Hirata-Tsuchiya et al., Mol Endocrinol 28(9):1460-1470 (2014)) and it would be interesting to determine if TUT7 regulates NFκB activity in osteoblasts. If confirmed, this may explain the decrease in RANKL expression seen in the TUT7KO mice. Whether TUT7 regulates RANKL mRNA expression directly or through uridylation of RANKL-related miRNAs is not clear and other studies will focus on TUT7-mediated regulation of RANKL expression and the results will be reported elsewhere.

We also found that absence of TUT7 enhances Osterix expression and activity in osteoblasts. Osterix is a master transcription factor critical for osteoblast differentiation and function. Zhou et al., Proc Natl Acad Sci USA., 107(29):12919-12924 (2010) The zinc finger proteins have been shown to have important functions in transcriptional and translational regulation and processing. Uridylation occurs pervasively on mRNAs and TUT4 and TUT7 (TUT4/7), were recently shown to be mRNA uridylation enzymes. Lim et al., Cell 159(6):1365-1376 (2014) Uridylation readily occurs on deadenylated mRNAs and are marked for degradation. However, it was found that in cells depleted of TUT4/7, the vast majority of mRNAs lose the oligo-U-tails, and their half-lives were extended. Therefore, it is tempting to speculate that physiologically TUT7 regulates Osterix expression in osteoblasts by uridylating Osterix mRNA and marking it for degradation. Thus, in the absence of TUT7 expression accumulation of Osterix mRNA and its translation proceeds without hindrance and this may explain the high levels of Osterix expression and activity in TUT7KO osteoblasts. It is also possible that TUT7 regulation of Osterix may be independent of its uridyltransferase activity similar to findings in the function of the closely related TUT4 gene. Blahna et al., J Biol Chem. 286(49):42381-42389 (2011) It will be interesting to determine the specific role of TUT7 in regulating cellular activities of bone cells through both its RNA tailing and non-nucleotidyl transferase mechanisms.

Our study is the first to explore the role of non-canonical PAPs in bone remodeling. We show that absence of TUT7 enhances bone mass by increasing osteoblast matrix mineralization and decreasing osteoclast differentiation in vivo. Furthermore, we show that TUT7 acts primarily in osteoblasts through regulation of Osterix and RANKL expression. Overall, we conclude that TUT7 is a negative regulator of bone homeostasis, and may be a potential therapeutic target for the treatment of osteoporosis and subchondral bone remodeling in osteoarthritis.

### Example 2: Human ZCCHC6 Ribonucleotidyl Transferase Regulates Interleukin-6 Expression is Osteoarthritic Chondrocytes

Interleukin-1β (IL-1β) is the major cytokine involved in cartilage catabolism in osteoarthritis (OA) and induces the expression of pro-inflammatory cytokine IL-6. IL-6 is known to induce the expression of MMP-13 and inhibit type-II collagen expression. Cytoplasmic RNA nucleotidyl transferases catalyze the addition of nucleotides to the 3′ end of mRNAs. However, the expression or role of RNA nucleotidyl transferases in regulating cytokine expression in OA is unknown. The aim of this study was to investigate whether RNA nucleotidyl transferase ZCCHC6, a recently identified member of the ribonucleotidyl transferases superfamily, is expressed in OA cartilage, identify the cytokines regulated by ZCCHC6 in chondrocytes, and whether ZCCHC6 is involved in the regulation of IL-6 expression in OA chondrocytes.

Chondrocytes were derived by enzymatic digestion of human cartilage obtained from OA patients (n=14) undergoing knee joint replacement. Chondrocytes were stimulated with IL-1β (5 ng/ml) or treated with Actinomycin D (5 μg/ml) or NFκB inhibitor SC514 (75 μM) or JNK inhibitor (25 μM). Total RNA from grounded cartilage and from chondrocytes was purified using Qiagen RNeasy kit (Qiagen). Reverse transcription was performed using the Quantitect Reverse Transcription kit and the ZCCHC6 or IL-6 mRNA was quantified using TaqMan assays. SiRNA-mediated depletion of ZCCHC6 in human chondrocytes was used to study the effect on inflammatory cytokine expression using a cytokine array (Ray Biotech). Protein expression of IL-6 was studied using Western immunoblotting and quantified by ELISA in culture supernatants. Results were derived using Sigma Plot 12.3 package and p<0.05 was considered significant.

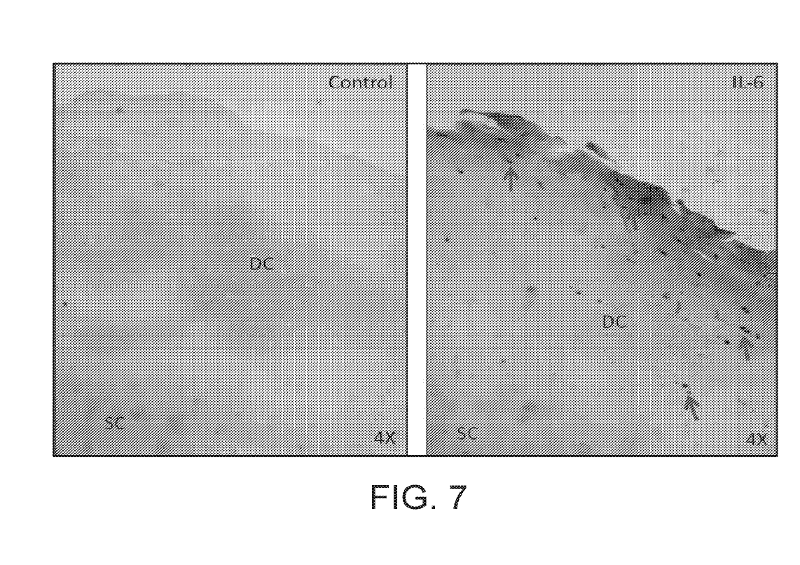

The results showed higher expression of nucleotidyl transferase ZCCHC6 in the damage cartilage compared to unaffected cartilage. See FIG. 7. Higher expression of IL-6 (6.0 fold±1.44) in damaged cartilage compared to smooth cartilage (n=3; p<0.05) from OA patients was also observed. We further demonstrate that IL-1β stimulation resulted in a significant increase in the expression of ZCCHC6 (11.3-fold±1.6) and the mRNA of the inflammatory cytokine IL-6 (4956-fold±40.6) in human chondrocytes (n=4; p<0.05). Similar increase in the protein expression of both ZCCHC6 and IL-6 was also observed. Depletion of ZCCHC6 significantly decreased the expression of IL-6 mRNA (˜77-95%) and protein in IL-1β-stimulated chondrocytes and IL-6 mRNAs in IL-1β-stimulated chondrocytes treated with ZCCHC6 siRNA had shorter poly-A tails (n=3; p<0.05). Cell supernatants from control or ZCCHC6 siRNA treated chondrocytes stimulated with IL-1β were analyzed using a cytokine array. A subset of cytokines including IL-6 was substantially decreased by loss of ZCCHC6. Our results also showed that the IL-1β-induced activation of NF-κB has no role in regulation of ZCCHC6 expression in OA chondrocytes and ZCCHC6 expression was regulated by JNK-MAPKs (n=3; p<0.05). Additionally, OA chondrocytes transfected with ZCCHC6 siRNA also showed a decrease (86%) in constitutive IL-6 mRNA expression (n=3; p<0.05).

Taken together, the results demonstrate for the first time that ZCCHC6 is highly expressed in damaged human cartilage compared from OA patients. Furthermore, ZCCHC6 modulates IL-6 expression in human chondrocytes at the post-transcriptional level by influencing cytokine mRNA stability. These results identify ZCCHC6 as a possible therapeutic target for the treatment of OA.

### Example 3: ZCCHC6 Effect on IL-6 Production

Interleukin-1β stimulates the expression of several inflammatory mediators including IL-6 which play an important role in the pathogenesis of osteoarthritis. Interleukin-6 is a pro-inflammatory cytokine that activates the transcription of its target genes via formation of an IL-6 receptor complex involving a membrane bound IL-6 receptor (IL-6R), soluble IL-6R (sIL-6R) and gp130 followed by activation of STAT1/STAT3 pathway. Guerne et al. showed the release of IL-6 from synoviocytes in response to IL-1 and detected IL-6 activity in synovial fluids obtained from osteoarthritis (OA) patients. 11. Guerne et al., J. Clin. Invest. 83: 585-592 (1989). Later, the same group showed the release of IL-6 from human chondrocytes in response to IL-1 and TNF-α. 12. Guerne et al., J. Immunol. 144, 499-505 (1992). Besides cytokines, type II collagen and PGE₂ also stimulate the production of IL-6 from human chondrocytes. IL-6 is elevated in synovial fluid of patients with OA, and has also been shown to act as a crucial mediator of MMP13 levels in HIF-2α-induced experimental OA in mice. Ryu et al., Arthritis Rheum., 63:2732-2743 (2011). IL-6 is reported to exert its effect on catabolic mechanism in cartilage by upregulating the expression of MMP-13 in combination with IL-1β and Oncostatin M (OSM) in human and bovine cartilage explants (Rowan et al., Arthritis Rheum. 44: 1620-1632 (2001)) and affect the anabolic process in cartilage by inhibiting the expression of type II collagen. 20. Porée et al., J. Biol. Chem. 283:4850-4865 (2008). A prospective population study on a cohort of British women showed a correlation of higher BMI and elevated serum levels of IL-6 with development of radiographic knee OA. 21. Livshits et al., Arthritis Rheum. 60 2037-2045 (2009).

In our preliminary studies (Example 2) employing standard immunohistochemical techniques we found that chondrocytes present in damaged cartilage express IL-6 while little or no IL-6 expressing cells were found in the cartilage area that stained positive with Safranin-O. Thus, taken together there exists a strong correlation between the expression of IL-6 and cartilage degradation in human OA patients and in animal models of OA. Recently, a novel post-transcriptional regulation of cytokine expression mediated by the 3′ end modifications of miRNAs that target IL-6 mRNA by a member of TUTase family of enzymes has been shown in cancer but post-transcriptional regulation of IL-6 expression and expression and function of TUTases in OA has not been explored in detail. In our preliminary studies, we identify a novel TUTase ZCCHC6 that is differentially expressed in human OA cartilage and chondrocytes and its expression is modulated by IL-1β. We therefore propose to investigate whether ZCCHC6 post-transcriptionally regulates IL-6 expression in human chondrocytes stimulated with IL-1β and we will determine the impact of zcchc6 deletion on IL-6 expression and disease severity using a zcchc6 knock-out mouse.

IL-1β-Induces Expression and Production of IL-6 and ZCCHC6 by Human OA Chondrocytes In Vitro

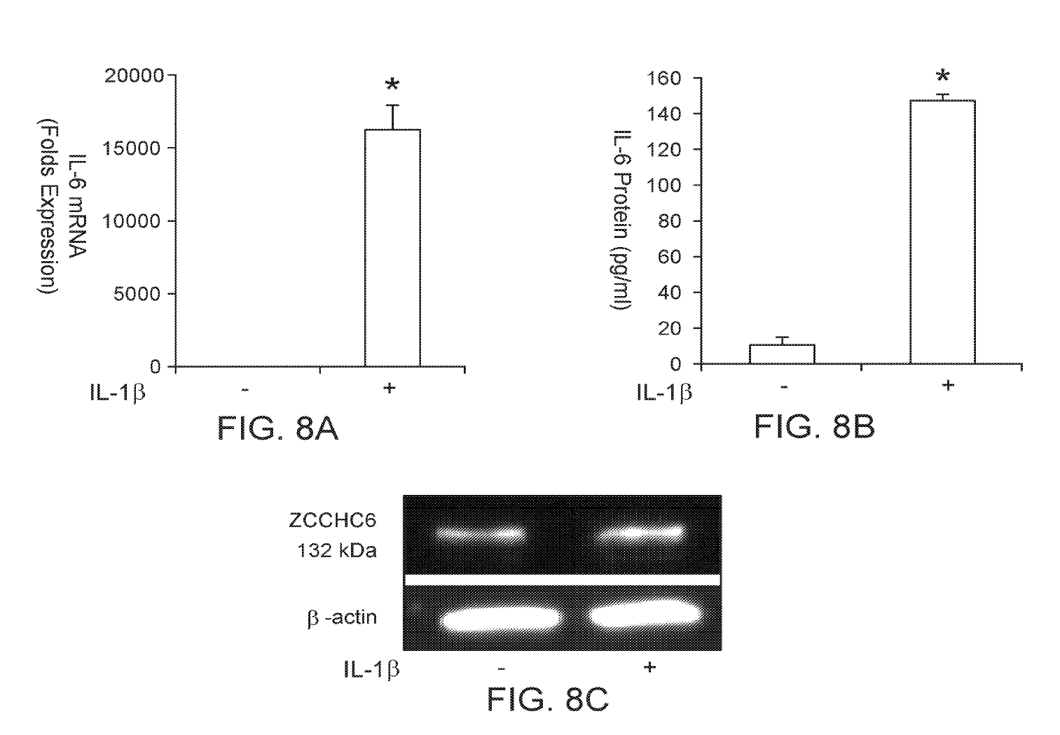

We next determined whether IL-1β induces expression of IL-6 correlates with the expression of ZCCHC6 in human OA chondrocytes. Human OA chondrocytes were cultured and treated with IL-1β as above and the mRNA expression of IL-6 was quantified by TaqMan assay and secreted IL-6 was quantified using an ELISA kit specific for human IL-6 (R&D Systems, Catalog No. D0650, sensitivity of 2.17 pg IL-6/ml). ZCCHC6 protein expression was determined by Western immunoblotting using an antibody specific for the N-terminus of the protein (Santa Cruz, sc-137947). These results, shown in FIG. 8, demonstrate that in untreated chondrocytes expression of IL-6 mRNA was barely detectable and negligible amounts of IL-6 were secreted into the medium. In contrast, stimulation of human chondrocytes with IL-1β resulted in high levels of expression of IL-6 mRNA and these cells also secreted large amounts of IL-6 in the culture medium (FIG. 8B). Also, in chondrocytes stimulated with IL-1β, expression of ZCCHC6 protein was highly induced (FIG. 8C). These data indicate that IL-1β is a potent inducer of both IL-6 and ZCCHC6 mRNA and protein expression in human OA chondrocytes. Furthermore, OA chondrocytes that showed high levels of ZCCHC6 expression also produced more IL-6 in the culture supernatant.

siRNA-Mediated Knockdown of ZCCHC6 in Human Chondrocytes Inhibited IL-6 Expression

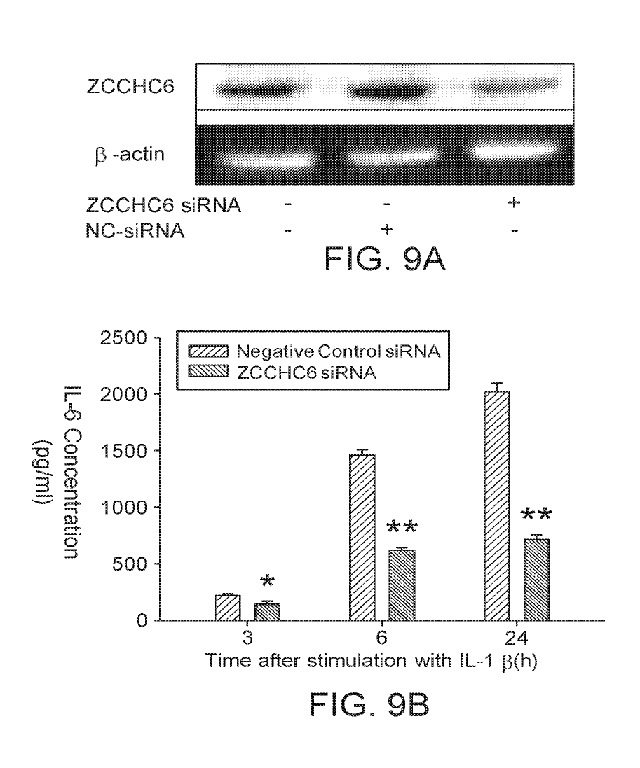

To determine if ZCCHC6 plays a direct role in IL-6 expression in OA chondrocytes, human OA chondrocytes were transfected with 100 nanomoles of ZCCHC6 specific targeting siRNAs (OnTarget Plust, SMARTPOOL siRNAs, Dharmacon) using the Amaxa system and effective knockdown relative to control, non-targeting siRNA was confirmed by immunoblotting using a ZCCHC6-specific antibody (Santa Cruz Biotechnology, sc-137947). Viability of transfected chondrocytes was determined by Trypan Blue exclusion assay and no significant effect on chondrocyte viability was observed. Transfection of human OA chondrocytes with the ZCCHC6 targeting SMARTPOOL siRNAs knocked down the ZCCHC6 protein expression by >75% in the transfected OA chondrocytes (FIG. 9A). To determine the role of ZCCHC6 in IL-6 expression, human OA chondrocytes with knocked down ZCCHC6 expression were stimulated with IL-1β and the secreted IL-6 protein in the culture supernatants was quantified using a sandwich ELISA assay kit (R&D Systems Cat# D6050). Compared to the levels detected in culture supernatants of chondrocytes transfected with non-targeting siRNAs, knockdown of ZCCHC6 resulted in a dramatic decrease in IL-6 protein secretion in culture supernatants of human OA chondrocytes transfected with ZCCHC6 targeting siRNAs (FIG. 9B). Importantly, siRNA-mediated knock down of ZCCHC11 expression had no effect on IL-6 protein expression in human chondrocytes. Taken together, these data indicate a correlation between the expression of ZCCHC6 and the expression of IL-6 in human OA chondrocytes.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A transgenic knockout non-human animal whose genome comprises a heterozygous disruption of the expression of at least one endogenous gene encoding zinc-finger, CCHC domain-containing protein 6 (ZCCHC6).

2. The transgenic knockout non-human animal of claim 1, wherein the non-human animal is a rodent.

3. The transgenic knockout non-human animal of claim 2, wherein the rodent is a mouse.

4. The transgenic knockout animal of claim 3, wherein expression of the ZCCHC6 is disrupted by recombination between the endogenous ZCCHC6 gene and a ZCCHC6 gene targeting vector comprising SEQ ID NO: 2.

5. A method of studying the role of zinc-finger, CCHC domain-containing protein 6 (ZCCHC6) in bone disease or development, comprising the steps of obtaining an animal model comprising a non-human animal having a heterozygous disruption of the expression of at least one endogenous gene encoding ZCCHC6, and determining differences in bone or cartilage characteristics, protein expression, or cytokine expression in the animal model in comparison to a corresponding wild-type non-human animal.

6. The animal method of claim 5, where the animal model is used to study bone disease.

7. The method of claim 6, wherein the bone disease is osteoarthritis.

8. The method of claim 5, wherein the animal model is used to study early post-natal bone development.

9. The method of claim 5, wherein the animal model is a rodent.

10. The method of claim 8, wherein the rodent is a mouse.

11. The method of claim 5, wherein the differences in protein expression or cytokine expression are evaluated in chondrocytes.

12. The method of claim 5, further comprising the step of administering ZCCHC6 protein to the animal model.
